SEQUENCE LISTING (8 sequences)

SEQ ID NO: 1 - DNA, 20 bp, Artificial Sequence
  Description of Artificial Sequence Synthetic primer
aaatctgctggggaccctac

SEQ ID NO: 2 - DNA, 20 bp, Artificial Sequence
  Description of Artificial Sequence Synthetic primer
tcccagattctgaaggcttg

SEQ ID NO: 3 - DNA, 21 bp, Artificial Sequence
  Description of Artificial Sequence Synthetic primer
agacactgcctgggtgctcct

SEQ ID NO: 4 - DNA, 23 bp, Artificial Sequence
  Description of Artificial Sequence Synthetic primer
gtttgatatttggtccttgtaag

SEQ ID NO: 5 - DNA, 19 bp, Artificial Sequence
  Description of Artificial Sequence Synthetic primer
gaaggtgaagttcggagtc

SEQ ID NO: 6 - DNA, 20 bp, Artificial Sequence
  Description of Artificial Sequence Synthetic primer
gaagatggtgatgggatttc

SEQ ID NO: 7 - DNA, 26 bp, Artificial Sequence
  Description of Artificial Sequence Synthetic primer
acgcgtatggcttcagaggcagcctg

SEQ ID NO: 8 - DNA, 25 bp, Artificial Sequence
  Description of Artificial Sequence Synthetic primer
gctagcttggtcttctggaagtaga

(19) United States
(12) Patent Application Publication
(10) Pub. No.: US 2019/0060406 A1
(43) Pub. Date: Feb. 28, 2019

(54) IL-1RA BASED COMPOSITIONS AND TREATMENTS

(71) Applicants: University of Southern California, Los Angeles, CA (US); Alfred E. Mann Institute for BioMedical Engineering at the University of Southern, Los Angeles, CA (US)

(72) Inventor: Songtao Shi, Thousand Oaks, CA (US)

(21) Appl. No.: 15/559,813

(22) PCT Filed: Mar. 22, 2016
(86) PCT No.: PCT/US16/23579

§ 371 Date: Sep. 19, 2017

Related U.S. Application Data
(60) Provisional application No. 62/138,233, filed on Mar. 25, 2015.

(51) Int. Cl. A61K 38/20; A61K 35/28; A61P 17/02; C07K 14/545; C12N 5/0775

(52) U.S. Cl. CPC A61K 38/2006; A61K 35/28; A61K 38/00; C07K 14/545; C12N 5/0668; A61P 17/02

(57) ABSTRACT

This disclosure relates to a stem cell treatment. This disclosure also relates to an IL-1RA treatment. This disclosure also relates to a composition comprising stem cells and preparation of this composition. This disclosure also relates to a composition comprising IL-1RA, an IL-1RA substitute, or a combination thereof. This disclosure also relates to preparation of this composition. This disclosure further relates to a wound treatment using a composition comprising IL-1RA, an IL-1RA substitute, or a combination thereof. This disclosure also further relates to a treatment to reduce scar formation by using a composition comprising IL-1RA, an IL-1RA substitute, or a combination thereof.

### CROSS-REFERENCE TO RELATED APPLICATION

This application claims the benefit of PCT application no. PCT/US2016/023579 entitled “IL-1RA Based Compositions and Treatments,” filed Mar. 22, 2016, attorney docket number 064693-0380, which claims the benefit of U.S. provisional patent application No. 62/138,233, entitled “IL-1RA Based Compositions and Treatments,” filed Mar. 25, 2015, attorney docket number 064693-0310, the entire content of which are incorporated herein by reference.

### STATEMENT REGARDING FEDERALLY SPONSORED RESEARCH

This invention was made with government support under Grant No. R01 DE017449 awarded by National Institutes of Health (NIH). The government has certain rights in the invention.

### BACKGROUND

### Technical Field

This disclosure relates to a stem cell treatment. This disclosure also relates to an IL-1RA treatment. This disclosure also relates to a composition comprising stem cells and preparation of this composition. This disclosure also relates to a composition comprising IL-1RA and preparation of this composition. This disclosure further relates to a wound treatment using stem cells and/or IL-1RA. This disclosure also further relates to a treatment to reduce scar formation by using a stem cell and/or IL-1RA.

### Description of Related Art

Wound healing is a sophisticated process of tissue repair involving multiple cell types and biological pathways. Mammalian adult cutaneous wound healing is mediated by a fibro-proliferative response with scar formation. Dysregulation of this interactive process (e.g., diabetes) may adversely result in delayed wound healing or hypertrophic scarring (Loots et al., 1998; Gurtner et al., 2008). Especially, if the inflammatory phase is prolonged, the pro-inflammatory mediators will direct the myofibroblasts and fibrocytes in larger scar formation (Rhett et al., 2008). These chronic wounds represent the most significant challenges in current clinical practice with over 50% of them being refractory to the treatment (Summer, 2012).

In contrast, early to mid-gestational fetal cutaneous wound healing is more akin to a scarless regeneration. Previous studies have shown that fetal wounds appear to have mild inflammation with fewer inflammatory cells and lower levels of inflammatory factors (Hu et al., 2014). Intriguingly, there is a common impression among clinicians that oral gingiva/mucosa wound heals faster and with minimal scar formation (Häkkinen et al., 2000) when compared to cutaneous wound healing. More importantly, the healing process shows a similar reduced inflammatory cell infiltration and proinflammatory factor production as the fetal wound healing (Szpaderska et al., 2003; Larjava et al., 2011). These evidences indicate that the outcome of wound healing process is largely affected by inflammatory regulation.

Mesenchymal stem cells (MSCs) are described to be a type of multipotent progenitor cells with ability to self-renewal and to differentiate into multiple cell lineages (Friedenstein et al., 1974; Prockop, 1997). Furthermore, MSCs are shown to have the capability to regulate immune response, which has provided a foundation for the use of MSCs in immune therapies (Uccelli et al., 2008; Le Blanc et al., 2008; Sun et al., 2009; Akiyama et al., 2012). It is generally agreed that local connective tissue fibroblasts are the major source involved in skin wound site repair (R. Higashiyama et al., 2011).

In addition, it is now well documented that the local MSCs such as the stem cells residing in the dermal sheath that surrounds the outside of the hair follicle can also differentiate into fibroblasts after a lesion (C. A. Jahoda et al., 2001 and 2003).

Recently, gingiva/mucosa-derived mesenchymal stem cells (GMSCs) were isolated and characterized. Interestingly, these cells have a distinct neural crest origin, share some traits with fetal cells and show potent immunoregulatory properties suggesting that they may possess specific therapeutic potential (Xu et al., 2013; Fournier et al., 2013).
Allan S M, Tyrrell P J, Rothwell N J. (2005) Interleukin-1 and neuronal injury. Nat Rev Immunol, 5(8): 629-640.
Loots M A, Lamme E N, Zeegelaar J et al. (1998) Differences in cellular infiltrate and extracellular matrix of chronic diabetic and venous ulcers versus acute wounds. J Invest Dermatol, 111:850-857.
Gurtner G C, Werner S, Barrandon Y, Barrandon Y, Longaker M T. (2008) Wound repair and regeneration. Nature, 453: 314-321.
Rhett J M, Ghatnekar G S, Palatinus J A, O'Quinn M, Yost M J, Gourdie R G. (2008) Novel therapies for scar reduction and regenerative healing of skin wounds. Trends Biotechnol, 26: 173-180.
Summer E H. (2012) Mesenchymal stem cells: a multimodality option for wound healing. Adv Wound Care, 1: 153-158.
Hu M S, Maan Z N, Wu J C, Rennert R C, Hong W X, Lai T S et al. (2014) Tissue engineering and regenerative repair in wound healing. Ann Biomed Eng, 42(7): 1494-1507.
Häkkinen L, Uitto V J, Larjava H. (2000) Cell biology of gingival wound healing. Periodontology 2000 24: 127-152.
Szpaderska A M, Zuckerman J D, DiPietro L A. (2003) Differential injury responses in oral mucosal and cutaneous wounds. J Dent Res 82: 621-626.
Glim J E, van Egmond M, Niessen F B, Everts V, Beelen R H (2013) Detrimental dermal wound healing: What can we learn from the oral mucosa? Wound Repair Regen 21: 648-660.
Mak K, Manji A, Gallant-Behm C, Wiebe C, Hart D A, et al. (2009) Scarless healing of oral mucosa is characterized by faster resolution of inflammation and control of myofibroblast action compared to skin wounds in the red Duroc pig model. J Dermatol Sci 56: 168-180.
Larjava H, Wiebe C, Gallant-Behm C, Hart D A, Heino J, Häkkinen L. (2011) Exploring scarless healing of oral soft tissues. J Can Dent Assoc 77: b18.
Dinarello C A. (2010) Anti-inflammatory Agents: Present and Future. Cell. 140: 935-950.
R. Higashiyama, S. Nakao, Y. Shibusawa, O. Ishikawa, T. Moro, K. Mikami, et al. Differential contribution of dermal resident and bone marrow-derived cells to collagen production during wound healing and fibrogenesis in mice. J Invest Dermatol. 131 (2011), pp. 529-536
C. A. Jahoda, J. Whitehouse, A. J. Reynolds, N. Hole. Hair follicle dermal cells differentiate into adipogenic and osteogenic lineages. Exp Dermatol. 12 (2003), pp. 849-859
C. A. Jahoda, A. J. Reynolds. Hair follicle dermal sheath cells: unsung participants in wound healing. Lancet, 358 (2001), pp. 1445-1448
M. F. Pittenger, A. M. Mackay, S. C. Beck, R. K. Jaiswal, R. Douglas, J. D. Mosca, et al. Multilineage potential of adult human mesenchymal stem cells. Science, 284 (1999), pp. 143-147
S. R. Opalenik, J. M. Davidson. Fibroblast differentiation of bone marrow-derived cells during wound repair. FASEB J, 19 (2005), pp. 1561-1563
N. C. Direkze, S. J. Forbes, M. Britten, T. Hunt, R. Jeffery, S. L. Preston, et al. Multiple organ engraftment by bone-marrow-derived myofibroblasts and fibroblasts in bone-marrow-transplanted mice. Stem Cells, 21 (2003), pp. 514-520
Friedenstein, A. J., R. K. Chailakhyan, N. V. Latsinik, A. F. Panasyuk, and I. V. Keiliss-Borok. 1974. Stromel cells responsible for transferring the microenvironment of the hemopoietic tissues. Cloning in vitro and retransplantation in vivo. Transplantation. 17: 331-40.
Prockop, D. J. 1997. Marrow stromal cells as stem cells for nonhematopoietic tissues. Science. 276: 71-4.
Uccelli, A., Moretta, L., Pistoia, V. (2008). Mesenchymal stem cells in health and disease. Nat Rev Immunol 8:726-736.
Le Blanc K, Frassoni F, Ball L, Locatelli F, Roelofs H, Lewis I, et al. (2008). Mesenchymal stem cells for treatment of steroid-resistant, severe, acute graft-versus-host disease: a phase II study. Lancet 371:1579-1586.
Sun L, Akiyama K, Zhang H, Yamaza T, Hou Y, Zhao S, et al. (2009). Mesenchymal stem cell transplantation reverses multiorgan dysfunction in systemic lupus erythematosus mice and humans. Stem Cells 27:1421-1432.
Akiyama K, Chen C, Wang D, Xu X, Qu C, Yamaza T, et al. (2012). Mesenchymal-stem-cell-induced immunoregulation involves FAS-ligand-/FAS-mediated T cell apoptosis. Cell Stem Cell 10:544-555.
Xu X, Chen C, Akiyama K, Chai Y, Le A D, Wang Z, et al. (2013). Gingivae contain neural-crest- and mesoderm-derived mesenchymal stem cells. J Dent Res. 92(9):825-832.
Fournier B P, Larjava H, Häkkinen L. (2013) Gingiva as a source of stem cells with therapeutic potential. Stem Cells Dev 22(24): 3157-3177.
Su W R, Zhang Q Z, Shi S H, Nguyen A L, Le A D (2011). Human gingiva derived mesenchymal stromal cells attenuate contact hypersensitivity via prostaglandin E(2)-dependent mechanisms. Stem Cells 29:1849-1860.
Tang L, Li N, Xie H, Jin Y (2011). Characterization of mesenchymal stem cells from human normal and hyperplastic gingiva. J Cell Physiol 226:832-842.
Zhang Q, Shi S, Liu Y, Uyanne J, Shi Y, Shi S, et al. (2009). Mesenchymal stem cells derived from human gingiva are capable of immunomodulatory functions and ameliorate inflammation-related tissue destruction in experimental colitis. J Immunol 183:7787-7798.
Zhang Q, Su W R, Shi S H, Wilder-Smith P, Xiang A P, Wong A, et al. (2010). Human gingiva-derived mesenchymal stem cells elicit polarization of m2 macrophages and enhance cutaneous wound healing. Stem Cells 28:1856-1868.
Garlanda C, Dinarello C A, Mantovani A. The interleukin-1 family: back to future. (2013) Immunity. 39(6): 1003-1018.
Muzio M, Polentarutti N, Sironi M, Poli G, De Gioia L, Intone M, et al. (1995) Cloning and Characterization of a New Isoform of the Interleukin 1 Receptor Antagonist. J Exp Med. 182(2): 623-628.
F. L. van de Veerdonk, A. K. Stoeckman, G. Wu, A. N. Boeckermann, T. Azam, M. G. Netea, L. A. Joosten, J. W. van der Meer, R. Hao, V. Kalabokis, C. A. Dinarello. (2012) IL-38 binds to the IL-36 receptor and has biological effects on immune cells similar to IL-36 receptor antagonist. Proc. Natl. Acad. Sci. USA, 109: 3001-3005.
Arend, W P. The balance between IL-1 and IL-1ra in disease. (2002) Cytokine Growth Factor Rev, 13:323-340.
Ishida Y, Kondo T, Kimura A, Matsushima K, Mukaida N. Absence of IL-1 receptor antagonist impaired wound healing along with aberrant NF-kappaB activation and reciprocal suppression of TGF-beta signal pathway. (2006) J Immunol, 176(9): 5598-5606.
Horai R, Saijo S, Tanioka H, Nakae S, Sudo K, Okahara A, et al. (2000) Development of chronic inflammatory arthropathy resembling rheumatoid arthritis in interleukin 1 receptor antagonist-deficient mice. J Exp Med. 191: 313-320.
M. J. Nicklin, D. E. Hughes, J. L. Barton, J. M. Ure, G. W. Duff. (2000) Arterial inflammation in mice lacking the interleukin 1 receptor antagonist gene. J Exp Med. 191: 303-312.
I. Aksentijevich, S. L. Masters, P. J. Ferguson, P. Dancey, J. Frenkel, A. van Royen-Kerkhoff, R. Laxer, U. Tedgard, E. W. Cowen, T. H. Pham, et al. (2009) An autoinflammatory disease with deficiency of the interleukin-1-receptor antagonist. N Engl J Med. 360: 2426-2437.
S. Reddy, S. Jia, R. Geoffrey, R. Loner, M. Suchi, U. Broeckel, et al. (2009) An autoinflammatory disease due to homozygous deletion of the IL1RN locus. N Engl J Med. 360: 2438-2444.
Pittenger, M. F. et al. (1999) Multilineage potential of adult human mesenchymal stem cells. Science 284, 143-147.
Phinney, D. G. and Prockop, D. J. (2007) Concise review: mesenchymal stem/multipotent stromal cells: the state of transdifferentiation and modes of tissue repair—current views. Stem Cells 25, 2896-2902.
Medzhitov, R. (2008) Origin and physiological roles of inflammation. Nature 454, 428-435
Luster, A. D. et al. (2005) Immune cell migration in inflammation: present and future therapeutic targets. Nat. Immunol. 6, 1182-1190
Eming, S. A. et al. (2007) Inflammation in wound repair: molecular and cellular mechanisms. J. Invest. Dermatol. 127, 514-525
Sasaki, M. et al. (2008) Mesenchymal stem cells are recruited into wounded skin and contribute to wound repair by transdifferentiation into multiple skin cell type. J. Immunol. 180, 2581-2587.
Wu, Y. et al. (2007) Mesenchymal stem cells enhance wound healing through differentiation and angiogenesis. Stem Cells 25, 2648-2659.
Chen, L. et al. (2008) Paracrine factors of mesenchymal stem cells recruit macrophages and endothelial lineage cells and enhance wound healing. PLoS ONE 3, e1886.
Gnecchi, M. et al. (2005) Paracrine action accounts for marked protection of ischemic heart by Akt-modified mesenchymal stem cells. Nat. Med. 11, 367-368.
Timmers, L. et al. (2011) Human mesenchymal stem cell-conditioned medium improves cardiac function following myocardial infarction. Stem Cell Res. 6, 206-214.
Shi Y, Su J, Roberts Al, Shou P, Rabson A B, Ren G. How mesenchymal stem cells interact with tissue immune responses. Trends Immunol 2012; 33: 136-143.
Aguilar S, Scotton C J, McNulty K, Nye E, Stamp G, Laurent G et al. Bone marrow stem cells expressing keratinocyte growth factor via an inducible lentivirus protects against bleomycin-induced pulmonary fibrosis. PLoS One 2009; 4: e8013.
Hung S P, Yang M H, Tseng K F, Lee O K. Hypoxia-induced secretion of TGF-beta 1 in mesenchymal stem cell promotes breast cancer cell progression. Cell Transplant 2012; 22: 1869-1882.
Krampera, M. et al. (2006) Role for interferon-gamma in the immunomodulatory activity of human bone marrow mesenchymal stem cells. Stem Cells 24, 386-398
Sheng, H. et al. (2008) A critical role of IFNgamma in priming MSC-mediated suppression of T cell proliferation through up-regulation of B7-H1. Cell Res. 18, 846-857
Mougiakakos, D. et al. (2011) The impact of inflammatory licensing on heme oxygenase-1-mediated induction of regulatory T cells by human mesenchymal stem cells. Blood 117, 4826-4835
Hübner G, Brauchle M, Smola H, Madlener M, Fässler R, Werner S. (1996) Differential regulation of pro-inflammatory cytokines during wound healing in normal and glucocorticoid-treated mice. Cytokine 8(7): 548-556.
Miller, L. S. et al. MyD88 mediates neutrophil recruitment initiated by IL-1R but not TLR2 activation in immunity against Staphylococcus aureus. (2006) Immunity 24, 79-91.
Dinarello, C. A. Biologic basis for interleukin-1 in disease. (1996) Blood 87, 2095-2147.
Chung, Y. et. al. Critical regulation of early Th17 cell differentiation by interleukin-1 signaling. (2009) Immunity 30, 576-587.
Acosta-Rodriguez, e. v., Napolitani, G., Lanzavecchia, A. & Sallusto, F. Interleukins 1β and 6 but not transforming growth factor-β are essential for the differentiation of interleukin 17-producing human T helper cells. (2007) Nat. Immunol. 8: 942-949.
Greenfeder, S. A. et al. Molecular cloning and characterization of a second subunit of the interleukin 1 receptor complex. (1995) J. Biol. Chem. 270, 13757-13765.
Arend, W. P., Malyak, M., Guthridge, C. J. & Gabay, C. Interleukin-1 receptor antagonist: role in biology. (1998) Annu. Rev. Immunol. 16, 27-55.
Palmer, G., Talabot-Ayer, D., Kaya, G. & Gabay, C. Type I IL-1 receptor mediates IL-1 and intracellular IL-1 receptor antagonist effects in skin inflammation. (2007) J. Invest. Dermatol. 127, 1938-1946.
Tibbetts, M. D., Zheng, L. & Lenardo, M. J. The death effector domain protein family: regulators of cellular homeostasis. (2003) Nature Immunol. 4, 404-409.
Brint E, O'Callaghan G, Houston A. (2013) Life in the fas lane: differential outcomes of fas signaling. Cell Mol Life Sci, 70:4085-4099.
Trauth B C, Klas C, Peters A M, Matzku S, Moller P, Falk W, Debatin K M, Krammer P H (1989) Monoclonal antibody-mediated tumor regression by induction of apoptosis. Science 245:301-305
Yonehara S, Ishii A, Yonehara M (1989) A cell-killing mono-clonal antibody (anti-Fas) to a cell surface antigen co-downregulated with the receptor of tumor necrosis factor. J Exp Med 169:1747-1756
Locksley R M, Killeen N, Lenardo M J (2001) The TNF and TNF receptor superfamilies: integrating mammalian biology. Cell 104:487-501
Scott F L, Stec B, Pop C, Dobaczewska M K, Lee J J, Monosov E, Robinson H, Salvesen G S, Schwarzenbacher R, Riedl S J (2009) The Fas-FADD death domain complex structure unravels signaling by receptor clustering. Nature 457:1019-1022
Scaffidi C, Fulda S, Srinivasan A, Friesen C, Li F, Tomaselli K J, Debatin K M, Krammer P H, Peter M E (1998) Two CD95 (APO-1/Fas) signaling pathways. EMBO J 17:1675-1687
Kovacs B, Tsokos G C (1995) Cross-linking of the Fas/APO-1 antigen suppresses the CD3-mediated signal transduction events in human T lymphocytes. J Immunol 155:5543-5549
Alderson M R, Armitage R J, Maraskovsky E, Tough T W, Roux E, Schooley K, Ramsdell F, Lynch D H (1993) Fas transduces activation signals in normal human T lymphocytes. J Exp Med 178:2231-2235
Freiberg R A, Spencer D M, Choate K A, Duh H J, Schreiber S L, Crabtree G R, Khavari P A (1997) Fas signal transduction triggers either proliferation or apoptosis in human fibroblasts. J Invest Dermatol 108:215-219
Chen L, Park S M, Tumanov A V, Hau A, Sawada K, Feig C, Turner J R, Fu Y X, Romero I L, Lengyel E, Peter M E (2010) CD95 promotes tumour growth. Nature 465:492-496
Desbarats J, Newell M K (2000) Fas engagement accelerates liver regeneration after partial hepatectomy. Nat Med 6:920-923
Reinehr R, Sommerfeld A, Haussinger D (2008) CD95 ligand is a proliferative and antiapoptotic signal in quiescent hepatic stel late cells. Gastroenterology 134:1494-1506
Lambert C, Landau A M, Desbarats J (2003) Fas-beyond death: a regenerative role for Fas in the nervous system. Apoptosis 8:551-562
Barnhart B C, Legembre P, Pietras E, Bubici C, Franzoso G, Peter M E (2004) CD95 ligand induces motility and invasiveness of apoptosis-resistant tumor cells. EMBO J 22:22
Li H, Fan X, Stoicov C, Liu J H, Zubair S, Tsai E, Ste Marie R, Wang T C, Lyle S, Kurt-Jones E, Houghton J (2009) Human and mouse colon cancer utilizes CD95 signaling for local growth and metastatic spread to liver. Gastroenterology 137:934-944
Trauzold A, Roder C, Sipos B, Karsten K, Arlt A, Jiang P, Mar-tin-Subero J I, Siegmund D, Muerkoster S, Pagerols-Raluy L, Siebert R, Wajant H, Kalthoff H (2005) CD95 and TRAF2 pro-mote invasiveness of pancreatic cancer cells. Faseb J 19:620-622
Letellier E, Kumar S, Sancho-Martinez I, Krauth S, Funke-Kaiser A, Laudenklos S, et al. (2010) CD95-ligand on peripheral myeloid cells activates Syk kinase to trigger their recruitment to the inflammatory site. Immunity 32:240-252
Zhang Y, Liu Q, Zhang M, Yu Y, Liu X, Cao X (2009) Fas signal promotes lung cancer growth by recruiting myeloid-derived suppressor cells via cancer cell-derived PGE2. J Immunol 182:3801-3808
Palao G, Santiago B, Galindo M A, Rullas J N, Alcami J, Ramirez J C, Pablos J L (2006) Fas activation of a proinflammatory program in rheumatoid synoviocytes and its regulation by FLIP and caspase 8 signaling. Arthritis Rheum 54:1473-1481
Ma Y, Liu H, Tu-Rapp H, Thiesen H J, Ibrahim S M, Cole S M, Pope R M (2004) Fas ligation on macrophages enhances IL-1R1-Toll-like receptor 4 signaling and promotes chronic inflammation. Nat Immunol 5:380-387
Eramo A, Sargiacomo M, Ricci-Vitiani L, Todaro M, Stassi G, et al. (2004) CD95 death-inducing signaling complex formation and internalization occur in lipid rafts of type I and type II cells. Eur J Immunol 34: 1930-1940
Gajate C, Mollinedo F (2001) The antitumor ether lipid ET-18-OCH(3) induces apoptosis through translocation and capping of Fas/CD95 into membrane rafts in human leukemic cells. Blood 98: 3860-3863.
Zhu X D, Zhuang Y, Ben J J, Qian L L, Huang H P, et al. (2011) Caveolae-dependent endocytosis is required for class A macrophage scavenger receptor-mediated apoptosis in macrophages. J Biol Chem 286: 8231-8239.
Guo Y H, Hernandez I, Isermann B, Kang T B, Medved L, et al. (2009) Caveolin-1-dependent apoptosis induced by fibrin degradation products. Blood 113: 4431-4439.
Parton R G, del Pozo M A. Caveolae as plasma membrane sensors, protectors and organizers. (2013) Nat Rev Mol Cell Biol, 14(2): 98-112.
Cao H, Courchesne W E, Mastick C C. (2002) A phosphotyrosine-dependent protein interaction screen reveals a role for phosphorylation of caveolin-1 on tyrosine 14: recruitment of C-terminal Src kinase. J Biol Chem 277: 8771-8774.
del Pozo, M. A. et al. Phospho-caveolin-1 mediates integrin-regulated membrane domain internalization. (2005) Nat Cell Biol, 7: 901-908.
Wrana J L, Attisano L, Cárcamo J, Zentella A, Doody J, Laiho M et al. TGF beta signals through a heteromeric protein kinase receptor complex. (1992) Cell. 71(6): 1003-1014.
Cahill D P, Lengauer C, Yu J, Riggins G J, Willson J K, Markowitz S D, et al. Mutations of mitotic checkpoint genes in human cancers. (1998) Nature. 392(6673): 300-303.
Wang Y, Chen X, Cao W, Shi Y. Plasticity of mesenchymal stem cells in immunomodulation: pathological and therapeutic implications. (2014) Nat Immunol. 15(11): 1009-1016.
Valadi H, Ekström K, Bossios A, Sjöstrand M, Lee J J, Lötvall J O. Exosome-mediated transfer of mRNAs and microRNAs is a novel mechanism of genetic exchange between cells. (2007) Nat Cell Biol. 9: 654-659.
Méndez-Ferrer, S. et al. Mesenchymal and haematopoietic stem cells form a unique bone marrow niche. Nature 466, 829-834 (2010).
Sutton R B, Fasshauer D, Jahn R, Brunger A T (1998). “Crystal structure of a SNARE complex involved in synaptic exocytosis at 2.4 Å resolution”. Nature 395 (6700): 347-353.

The entire contents of above publications are incorporated herein by reference.

### SUMMARY

This disclosure relates to a stem cell treatment. This disclosure also relates to an IL-1RA treatment. This disclosure also relates to a composition comprising stem cells and to the preparation of this composition. This disclosure also relates to a composition comprising IL-1RA and to the preparation of this composition. This disclosure further relates to a wound treatment using stem cells and/or IL-1RA. This disclosure also further relates to a treatment to reduce scar formation by using a stem cell and/or IL-1RA. A composition comprising a stem cell that may treat a wound. Preferably, the stem cells compositions are prepared from multipotent or pluripotent stem cells, and not from totipotent stem cells. More preferably, the stem cell compositions are prepared from multipotent stem cells, and not from pluripotent or totipotent stem cells.

As described and exemplified herein, certain stem cells contain intracellular IL-1RA, and can release the intracellular IL-1RA in response to stimuli (e.g., pro-inflammatory mediators) that are present at the site of a would. Thus, IL-1RA can be administered to a subject in need thereof by administering stem cells that contain intracellular IL-1RA to the subject. In some aspects, the disclosure relates to a composition that may comprise IL-1RA in which the composition contains a stem cell with intracellular IL-1RA.

The composition may comprise IL-1RA, which may treat a wound. This composition may improve wound healing. The composition may also comprise IL-1RA substitute, which may treat a wound. An example of an IL-1RA substitute may be anakinra, which is a recombinant, nonglycosylated form of the human IL-1RA. Anakinra differs from native human IL-1RA in that it has the addition of a single methionine residue at its amino terminus. It may be produced by recombinant DNA technology using an E coli bacterial expression system. The composition may comprise IL-1RA substitute with a concentration, for example, in the range of 1 mg per mL to 1,000 mg per mL of the composition; or 10 mg per mL to 200 mg per mL of the composition. The composition may comprise IL-1RA substitute with a concentration, for example, in the range of 1 mg per gram to 1,000 mg per gram of the composition; or 10 mg per gram to 200 mg per gram of the composition.

The composition may comprise a stem cell and may reduce scar formation when the composition is administered to treat a wound (e.g. applied to a wound). The composition may also comprise IL-1RA and may reduce scar formation when the composition is administered to treat a wound (e.g., applied to a wound).

The composition may comprise stem cells, for example, in the form of a cell culture. The composition may be a drug or a biologic formulation. The stem cell may be any stem cell. The stem cell may comprise gingiva derived mesenchymal stem cell (GMSC).

The wound may be any injury to any living tissue. An example of tissue may be skin. Examples of injury may be caused by trauma, such as a cut, blow, or other impact, including accidental and surgical reasons. The living tissue may be a living tissue of a mammal. The mammal may be a human. The mammal may be a non-human animal.

The treatment may comprise administering one or more compositions using any suitable methods, such as systemic infusion, localized application (including topical application), or other suitable means of formulation and delivery.

The preparation of a composition comprising a stem cell may comprise obtaining a tissue, separating the tissue into cells, identifying and/or isolating stem cells (e.g., by sorting), and preparing a composition comprising the stem cells.

Other exemplary embodiments are as follows.

A composition may comprise an IL-1RA substitute and at least one human stem cell that expresses IL-1RA. This composition may comprise an effective amount of the IL-1RA substitute such that the composition is effective in a treatment of a wound. The composition may comprise an effective amount of the IL-1RA substitute such that the composition is effective in reducing scar formation when the composition is applied to a wound. The composition may comprise an effective amount of human stem cells that are effective in a treatment of a wound. The composition may comprise an effective amount of human stem cells such that the composition is effective in reducing scar formation when the composition is applied to a wound. The composition may comprise an effective amount of the IL-1RA substitute and an effective amount of human stem cells such that the composition is effective in a treatment of a wound. The composition may comprise the IL-1RA substitute and an effective amount of human stem cells such that the composition is effective in reducing scar formation when the composition is applied to a wound. The stem cell may comprise a human mesenchymal stem cell. The human stem cell may comprise a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof. The human stem cell may comprise a human gingiva-derived mesenchymal stem cell. The IL-1RA substitute may be anakinra.

A method of treating a human subject may comprise administering an effective amount of a composition comprising human IL-1RA to the human subject, and thereby treating a wound of the human subject. The human IL-1RA may be derived from at least one human stem cell. The composition may comprise an effective amount of the human IL-1RA such that the composition is effective in the treatment of the wound. The human stem cell may comprise a mesenchymal stem cell. The human stem cell may comprise a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof. The human stem cell may comprise a human gingiva-derived mesenchymal stem cell.

A method of treating a human subject may comprise administering an effective amount of a composition comprising an IL-1RA substitute to the human subject and thereby treating a wound of the human subject. The composition may comprise an effective amount of the IL-1RA substitute such that the composition is effective in the treatment of the wound. The composition may comprise an effective amount of the IL-1RA substitute such that the composition is effective in reducing scar formation when the composition is applied to the wound. The IL-1RA substitute may be anakinra.

A method of treating a human subject may comprise administering an effective amount of a composition comprising an IL-1RA substitute and at least one human stem cell that expresses IL-1RA to the human subject, and thereby treating a wound of the human subject. The composition may comprise an effective amount of the IL-1RA substitute such that the composition is effective in the treatment of the wound. The composition may comprise an effective amount of the IL-1RA substitute such that the composition is effective in reducing scar formation when the composition is applied to the wound. The composition may comprise an effective amount of human stem cells that are effective in the treatment of the wound. The composition may comprise an effective amount of human stem cells such that the composition is effective in reducing scar formation when the composition is applied to the wound. The composition may comprise an effective amount of the IL-1RA substitute and an effective amount of the human stem cells such that the composition is effective in the treatment of the wound. The composition may comprise the IL-1RA substitute and an effective amount of human stem cells such that the composition is effective in reducing scar formation when the composition is applied to the wound. The human stem cell may comprise a human mesenchymal stem cell. The human stem cell may comprise a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof. The human stem cell may comprise a human gingiva-derived mesenchymal stem cell. The IL-1RA substitute may be anakinra.

A composition may comprise human IL-1RA. The human IL-1RA may be derived from at least one human stem cell. The composition may comprise an effective amount of the human IL-1RA such that the composition is effective in a treatment of a wound. The composition may comprise an effective amount of the human IL-1RA such that the composition is effective in reducing scar formation when the composition is applied to a wound. The human stem cell may comprise a mesenchymal stem cell. The human stem cell may comprise a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof. The human stem cell may comprise a human gingiva-derived mesenchymal stem cell.

The composition may further comprise a human stem cell. The human stem cell may comprise a human mesenchymal stem cell. The human stem cell may comprise a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof. The human stem cell may comprise a human gingiva-derived mesenchymal stem cell.

The composition may further comprise a stem cell culture. The stem cell culture may comprise a human mesenchymal stem cell. The stem cell culture may comprise a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof. The stem cell culture may comprise a human gingiva-derived mesenchymal stem cell.

The wound may be any injury to any living tissue of a mammal. The tissue may be skin. The injury may be caused by a cut or blow. The mammal may be a human. The mammal may be a non-human animal.

Any combination of features disclosed above may be possible and thereby within the scope of this disclosure. For example, any combination of above products (e.g. compositions) and methods (e.g. methods of treatment) is within the scope of this disclosure.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1. Cytokine array data showed that GMSCs expressed significant higher level of several cytokines including IL-1RA than SMSCs.

FIG. 2. (A) Western blotting showed SMSCs express less IL-1RA and FAS compared with GMSCs in wild-type mouse. (B) ELISA showed GMSCs released higher level of IL-1RA in culture supernatant compared with SMSCs. (C) Western blotting data showed that human GMSCs have the same trend as the mouse GMSCs, which expressed a higher FAS and IL-1RA in cytoplasm. (D) Western blotting showed that when use fas siRNA to block the FAS expression in human GMSCs, the IL-1RA level in human GMSCs will increase just as the observation in mouse GMSCs.

FIG. 3. (A) Immunofluorescence staining showed NESTIN+ mesenchymal stem cells were positive with IL-1RA expression in gingiva while not in skin. (B) Statistical analysis showed that in gingiva/mucosa, there were significantly high number of IL-1RA+ and NESTIN+ mesenchymal stem cells when compared with skin.

FIG. 4. (A) Wound (1.5 mm in diameter) healing delayed in IL-1RA KO mice at day 5. (B) Statistical analysis showed that the wound size was significantly larger in the IL-1RA KO mice than in the wild type control.

FIG. 5. (A) Schematic diagram of using IL-1RA neutralizing antibody to study the mucosa wound healing. (B) Pretreatment with IL-1RA neutralizing antibody compromised the mucosa wound healing in WT mice in multiple time points. (C) Statistical analysis showed that during each time course, the IL-1RA neutralizing antibody significantly delayed the wound healing process in wild type mice.

FIG. 6. Skin wound healed much faster in the WT compared with the IL-1RA KO mice at multiple time point.

FIG. 7. (A) GMSCs and SMSCs had same mRNA expression level of soluble IL-1RA. (B) GMSCs had a significantly higher mRNA expression level of intracellular IL-1RA when compared with SMSCs.

FIG. 8. (A) WT GMSCs secreted a higher level of IL-1RA in culture supernatant than Ipr GMSCs. (B) Western blotting showed that Ipr GMSCs remained more IL-1RA in cytoplasm. (C and D) When WT GMSCs with fas gene knock down, the IL-1RA was blocked in the cell with less secreted into the culture supernatant. (E and F) When Ipr GMSCs over expressed FAS, the IL-1RA secretion was enhanced with less remained in cells.

FIG. 9. Sucrose gradient centrifuge showed that IL-1RA was contained in the vesicle in very small size. The vesicle membrane associated protein 5 (VAMP5) showed a overlapping distribution with IL-1RA.

FIG. 10. (A-C) Immunoprecipitation experiment showed that phosphorylated CAVEOLIN-1 had more binding to SNAP25 and IL-1RA, inhibiting the IL-1RA from release.

FIG. 11. (A) Immunofluorescence staining showed co-localization of FAS, CAV-1 and SNAP25 on WT GMSCs membrane. (B-D) Knock down cav-1 and snap25 gene by siRNA showed the impaired release of IL-1RA into culture supernatant. (E) Sucrose gradient centrifuge showed that IL-1RA was contained in the vesicle of small size. (E and F) Western blotting showed that Ipr GMSCs had a higher level of phospho-CAVEOLIN-1, which was due to the FAS expression maintained the proper function of FAP-1. When knock down the fap-1 gene by siRNA, WT GMSCs also showed a higher phospho-CAVEOLIN-1 level.

FIG. 12. (A) Immunofluorescence staining showed Ipr GMSCs expressed higher level of phospho-CAVEOLIN-1. (B) Immunofluorescence staining showed alter the caveolae distribution in Ipr GMSCs.

FIG. 13. (A) When using the FAS neutralizing antibody to trigger FAS signaling, WT GMSCs also showed no difference in IL-1RA release. (B) Both WT and Ipr GMSCs expressed the calcium channel protein Cav 2.1 and 2.2 which are responsible for Ca+ controlled vesicle release. (C and D) When using chemical to block both of the calcium channel protein, WT GMSCs showed no difference in IL-1RA release. (E) When using TGF-β1 to stimulate GMSCs, the cells expressed a higher level of FAS and FAP-1. (F) Western blotting showed that after IFN-γ stimulation of the WT GMSCs, the cells showed a decreased level of phospho-CAVEOLIN1, which would further activate the secretory machinery system on cell membrane to release IL-1RA. (G) The ELISA data confirmed that when stimulated with IFN-γ, WT GMSCs had a higher IL-1RA concentration in culture supernatant. (H) When Ipr GMSCs were treated with IFN-γ, the phospho-CAVEOLIN1 level remained no change due to their lack of functional FAS on the membrane. (I) ELISA data showed that there was no significant concentration change of IL-1RA found in Ipr GMSCs culture supernatant.

FIG. 14. (A) Schematic diagram of using IL-1RA substitute to rescue the skin wound healing. (B) Skin wound healed much faster in the IL-1RA treated group compared with the saline treated control at multiple time point. (C) H&E staining showed that less inflammatory cells infiltration and better collagen deposition were observed in the IL-1RA treatment group.

FIG. 15. (A) SMSCs when treated with IL-1β had a elevated NF-κB signaling pathway leading to SMAD7 production, which inhibited the TGF-β pathway, while the IL-1RA can antagonize the IL-1β function. (B) TGF-β1 reaction plasmid showed the IL-1β largely inhibited the TGF-β1 effect on the SMSCs, However IL-1RA could significantly rescue the inhibition.

FIG. 16. (A) Less scar area and better aligned the collagen deposition were observed in the group of low dosage IL-1RA treated rabbit ear wound model. Although high dosage of IL-1RA treatment slightly compromised this effect, it still showed significantly better treatment effect when compared with the saline treated control group. (B) Statistical analysis showed that during each time course, the IL-1RA substitute significantly reduced the scar formation in rabbit ear model.

### DETAILED DESCRIPTION OF ILLUSTRATIVE EMBODIMENTS

Illustrative embodiments are now discussed. Other embodiments may be used in addition or instead. Certain details which may be apparent to a person of ordinary skill in the art or unnecessary may have been omitted from this disclosure to provide for a clear and concise disclosure. Conversely, some embodiments may be practiced without all of the details which are disclosed.

The following acronyms and abbreviations were used in this disclosure.

BMMSC: Bone marrow derived mesenchymal stem cells.

Con or Cont: Control.

Co-cul: Co-culture.

ELISA: Enzyme-linked immunosorbent assay technique.

Fas-L: Fas ligand.

GMSC: Gingiva derived mesenchymal stem cell.

g: Gram

IL: Interleukin.

IL-1: interleukin-1.

IL-10: interleukin 10.

IL-1RA: interleukin-1 receptor antagonist.

IFN-γ: interferon-γ.

kg: Kilogram.

mm: Millimeter.

mL: Milliliter.

MSC: Mesenchymal stem cell.

PBS: Phosphate-buffered saline.

PBMC: Peripheral blood mononuclear cells.

PCR: Polymerase chain reaction.

PD: Population doublings

RANKL: Receptor activator of nuclear factor κB ligand.

RT-PCT: Reverse transcription polymerase chain reaction.

SD: Standard deviation.

SMSC: Skin derived mesenchymal stem cell.

TNF: Tumor necrosis factor.

TUNEL: Terminal deoxynucleotidyl transferase-mediated UTP nick-end labeling.

μg: Microgram.

μm: Micrometer.

This disclosure in general relates to a treatment. This disclosure relates to a stem cell treatment. This disclosure also relates to an IL-1RA treatment. This disclosure also relates to a composition comprising stem cells and to methods for the preparation of this composition. This disclosure also relates to a composition comprising IL-1RA and to methods for the preparation of this composition. This disclosure further relates to a wound treatment using stem cells and/or IL-1RA. This disclosure also further relates to a treatment to reduce scar formation by using a stem cell and/or IL-1RA.

The treatment (i.e. method) includes, but not limited to, diagnosis, therapy, cure, healing, mitigation, or prevention of a wound, and/or cosmetic treatment of a mammal. Exemplary methods for treatment (e.g., administering the compositions disclosed herein) include systemic injection or infusion, localized application or injection (including topical application), or other suitable means of formulation and delivery.

This disclosure particularly relates to GMSCs that, when challenged by wound induced inflammatory cytokines, may increase their FAS expression leading to an elevated release of anti-inflammatory peptide(s) (e.g, via microvesicle mediated release) to counterbalance the local inflammatory environment, and thereby may improve wound healing and/or reduce scar formation.

The wound may be any injury to any living tissue. An example of tissue may be skin. Examples of injury may be caused by trauma, such as a cut, blow, or other impact, including accidental and surgical reasons. The living tissue may be a living tissue of a mammal.

The mammal may be a human. The mammal may be a non-human animal. For example, the mammal may be a non-human primate, a horse, a sheep, a cattle, a hog, a dog, a cat, and a goat.

This disclosure relates to a composition. The composition may be a cell culture. The composition may be a drug or a biologic formulation. The composition may be used in the treatment of a wound. An exemplary composition may comprise a stem cell, IL-1RA, an IL-1RA substitute, a stem cell and IL-1RA, a stem cell and an IL-1RA substitute, a stem cell and IL-1RA and an IL-1RA substitute, or a combination thereof. The IL-1RA can be intracellular IL-1RA present in a stem cell. In all embodiments of this disclosure, it is preferred that the compositions are prepared from multipotent or pluripotent stem cells, and not from totipotent stem cells. More preferably, the compositions are prepared from multipotent stem cells, and not from pluripotent or totipotent stem cells.

Stem cells, which are suitable for preparation of the exemplary composition, may be any stem cell of any mammal. For example, the stem cells may be stem cells of a mammal that undergoes the treatment (i.e. autologous stem cell treatment). Or, the stem cells may be stem cells of a mammal other than the mammal that undergoes the treatment (i.e. allogeneic stem cell treatment).

Examples of the stem cells may be embryonic stem cells, fetal stem cells, adult stem cells, amniotic stem cells, cord blood stem cells, induced pluripotent stem cells, or combinations thereof.

An example of the stem cells, that are useful in the preparation of the composition, may comprise a mesenchymal stem cell. Examples of mesenchymal stem cells may comprise bone marrow-derived mesenchymal cells, dental pulp stem cells, stem cells from human exfoliated deciduous teeth, periodontal ligament stem cells, dental follicle stem cells, tooth germ progenitor cells, stem cells from the apical papilla, oral epithelial progenitor/stem cells, gingiva-derived mesenchymal stem cells, periosteum-derived stem cells, salivary gland-derived stem cells, adipose derived mesenchymal stem cells, and combinations thereof. For example, the stem cells may be cultured mesenchymal stem cells, uncultured gingiva-derived mesenchymal stem cells, dental pulp stem cells, bone-marrow-derived stem cells, and combinations thereof.

One example of the stem cells, that is useful in the preparation of the composition, may comprise a bone marrow derived mesenchymal stem cell (BMMSC).

Another example of the stem cells, that is useful in the preparation of the composition, may comprise a gingiva derived mesenchymal stem cell (GMSC). The stem cells may be the isolated GMSCs. For isolation of the GMSCs, for example, see Le et al. in a United States patent application publication, entitled “Gingiva Derived Stem Cell and Its Application in Immunomodulation and Reconstruction,” Publication No. U.S. 2012/0128636 A1; Le et al. in a United States patent application publication, entitled “Pharmaceutical Compositions Comprising Gingiva Derived Mesenchymal Stem Cells and Methods of Treating Inflammation, Wound Healing and Contact Hypersensitivity,” Publication No. U.S. 2013/0295058 A1; and Shi et al. in a Patent Cooperation Treaty (PCT) patent application publication, entitled “A Composition of Mesenchymal Stem Cells,” Publication No. WO 2014210037 A2. The entire contents of these patent application publications are incorporated herein by reference.

This disclosure also relates to a method of preparation of the composition (“preparation method”).

One example of the preparation method may comprise obtaining a tissue, separating the tissue into cells, sorting a stem cell, and preparing a composition comprising the stem cells.

The tissue may be any tissue that contains a stem cell. For example, the tissue may comprise a mammalian tissue. In one example, the tissue may comprise a bone marrow tissue. In another example, the tissue may comprise a gingival tissue. The mammal may be a human. The mammal may be a non-human animal. For example, the mammal may be a non-human primate, a horse, a sheep, cattle, a hog, a dog, a cat, and a goat. The tissue may comprise a stem cell. The stem cells may be any stem cells. For example, the stem cells may be any stem cells disclosed above.

Another example of the preparation method may comprise obtaining a plurality of tissues, separating these tissues into cells, sorting the stem cells, and using the stem cells in the preparation of the composition. In this example, the plurality of tissues may comprise tissues obtained from different tissues of a mammal. For example, the first tissue may be a bone marrow tissue and the second tissue may be a gingival tissue.

The tissue may be obtained from a mammal that undergoes the treatment. In this method, the treatment may be an autologous stem cell treatment. The tissue may be obtained from a mammal other than the mammal that undergoes the treatment. In this method, the treatment may be an allogeneic stem cell treatment. Combination of said treatment methods may also be applied. For example, the treatment may comprise an autologous stem cell treatment and an allogeneic stem cell treatment.

The separating the tissue into cells may be done by a mechanical method, a chemical method, or a combination of a mechanical and a chemical method.

Examples of the mechanical method may be mincing, shredding, filtering, and the like. In other examples, the tissue may be separated into cells by using homogenizers, ultrasonicators, ball mills, and the like. A combination of these mechanical methods may also be used to have separated cells.

Examples of the chemical method may be digestion of the tissue by using acids, bases, and enzymes. For example, a collagenase and a dispase may be used to digest the tissue. The collagenase may be collagenase type I. The dispase may be dispase II. A combination of these chemical methods may also be used to have separated cells.

The preparation method may further comprise preparing cell suspensions from the digested tissue by using a mechanical method. An example of such method may be filtering the digested tissue to obtain cell suspensions. The cell suspensions may be single-cell suspensions. For example, single-cell suspensions may be obtained by passing the digested gingival tissue through a 70-micrometer trainer.

Culturing the separated cells may comprise providing a solid surface, seeding the cells on the solid surface, culturing the seeded cells, and thereby obtaining a culture comprising cells that may be adherent to the solid surface (“adherent cells”) and cells that may not be adherent to the solid surface (“non-adherent cells”).

The solid surface may be a surface of any solid article suitable for culturing cells. For example, it may be a wall of a culture vessel. The culture vessel may be any desired culture vessel. For example, the culture vessel may be a petri dish or a cell-culture dish. The solid article may also be a bead or a particle. The solid article may have any desired size. For example, it may be a nano-particle.

The cell may be seeded using a solution. The solution may comprise a medium suitable for culturing the mammalian cell. An example of such medium may be a-MEM manufactured by Invitrogen (Carlsbad, Calif.). The solution may further comprise fetal bovine serum (FBS), L-glutamine, 2-mercaptoethanol, penicillin, and streptomycin.

The preparation method may further comprise eliminating from the culture the cells that are not adherent to the solid surface. For example, the culture may be washed using PBS or other suitable buffer to eliminate from the culture the cells that are not adherent to the solid surface.

The adherent cells may further be cultured, for example, in the same conditions disclosed above.

The preparation method may further comprise dissociating from the solid surface the cells that may be adherent to the solid surface. The adherent cells may be dissociated from the solid surface by using an enzyme. The enzyme, for example, may be trypsin.

The preparation method may further comprise expanding the cultured cells. For example, the expanding the cultured cells may comprise doubling stem cells by repetitively re-seeding (passaging) them using the preparation methods disclosed above.

As disclosed in the above examples, the preparation method may comprise separating the tissue into cells and sorting the stem cells. Some examples of suitable methods for separating the tissue into cells and the sorting the stem cells have been previously disclosed. For example, for separating gingival tissue into cells, and sorting and isolating GMSCs, see Le et al. in a United States patent application publication, “Gingiva Derived Stem Cell and Its Application in Immunomodulation and Reconstruction” U.S. 2012/0128636 A1; and Shi et al. in a Patent Cooperation Treaty (PCT) patent application serial number PCT/US14/43918, “A Composition of Mesenchymal Stem Cells”. The entire content of these patent applications are incorporated herein by reference.

The preparation method may further comprise culturing the separated cells before sorting the stem cells. The sorting may comprise sorting cells that are positive for a stem cell marker, e.g., using an antibody labeled with a suitable label such as fluorescein isothiocyanate.

This disclosure also relates to a method of administration of the composition comprising a stem cell, IL-1RA, an IL-1RA substitute, a stem cell and IL-1RA, a stem cell and an IL-1RA substitute, a stem cell and IL-1RA and an IL-1RA substitute, or a combination thereof (“administration method”). The IL-1RA can be intracellular IL-1RA present in a stem cell. The administration method may comprise any method suitable for administration of a cell or a tissue. The administration method may comprise any method suitable for administration of IL-1RA. The composition comprising the stem cells and/or IL-1RA suitable for the treatment purposes may be administered (or delivered) in various ways. For example, they may be infused, injected at various sites, or surgically implanted. In some embodiments, the method may comprise administration of a stem cell and an IL-1RA substitute, either as component of a single composition or as separate compositions. When administered as separate compositions, the compositions are administered to provide for overlap in the therapeutic activity of the stem cell and the IL-1RA substitute. As described herein, the stem cell can include intracellular IL-1RA which can be released when the stem cell encounters pro-inflammatory stimuli (e.g., pro-inflammatory cytokines).

Suitable compositions include, for example, a stem cell and/or IL-1RA suspension in a liquid (preferably aqueous) medium, or the stem cells in aggregated form with or without solid supports or encapsulating materials. The liquid medium may be a pharmaceutically acceptable liquid medium. The liquid medium may be suitable for injection. Suitable liquid media are generally well known and include, for example, normal saline (with or without glucose and/or potassium), Ringer's solution, Lactated Ringer's solution, and Hartmann's solution.

The composition may be administered in an amount effective to treat a mammal. The amount of the stem cells, IL-1RA, IL-1RA substitute, or a combination thereof in the composition (“dose”) may be an amount effective to treat a mammal. “A combination thereof” here means any combination of the stem cell, IL-1RA, IL-1RA substitute. Examples of such combinations are disclosed above. The amount and/or concentration of stem cells, IL-1RA, IL-1RA substitute, or a combination thereof in the composition can be selected to provide for convenient administration of an amount (of the composition that is acceptable for the mammal being treated). For example, a liquid composition that contain a high concentration of the stem cells, IL-1RA, IL-1RA substitute, or a combination thereof is suitable for injection of a relatively small volume into solid tissue, while a liquid composition that contains a lower concentration of the stem cells, IL-1RA, IL-1RA substitute, or a combination thereof may be advantageous for administration by intravenous infusion. Suitable liquid compositions can contain, for example, about 1×10¹ stem cells/mL to about 1×10¹⁰ stem cells/mL, or about 1×10⁵ stem cells/mL to about 1×10⁷ stem cells/mL, or about 1×10⁶ stem cells/mL to 5×10⁶ stem cells/mL. Aggregated or solid compositions can contain, for example, about 1×10¹ stem cells/mg to about 1×10¹⁰ stem cells/g.

The composition may further comprise a carrier. The carrier may be suitable to host the stem cells and/or IL-1RA. Example of the carrier may be in the form of matrices, tissues, fibers, beads, or other materials.

The composition may comprise stem cells in an amount in the range of 1×10¹ stem cells/kg to 1×10¹⁰ stem cells/kg, or 1×10⁵ stem cells/kg to 1×10⁷ stem cells/kg, or 1×10⁶ stem cells/kg to 5×10⁶ stem cells/kg.

For therapeutic purposes, an effective amount of the composition is administered to a mammal in need thereof. An “effective amount” in an amount that produces the desired effect under the conditions of administration, for example, an amount sufficient to treat a wound, inhibit inflammation, inhibit an immune or autoimmune response, inhibit bone loss, inhibit tumor growth or metastisis, promote wound healing, or promote colony formation, osteogenesis or adipogenesis. The exact dosage of stem cells and/or IL-1RA to be administered may dependent upon a variety of factors, including the age, weight, and sex of the mammal, the disease being treated, and the extent and severity thereof. A clinician of ordinary skill can determine the appropriate dose and method for administration based on these and other considerations.

Any combination of products such as compositions comprising stem cells and/or IL-1RA, methods of their preparation, and methods of their use that are described herein may also be made and followed.

Other exemplary embodiments of this disclosure are as follows.

### Example 1. IL-1RA May Play a Critical Anti-Inflammatory Role in Oral Wound Healing

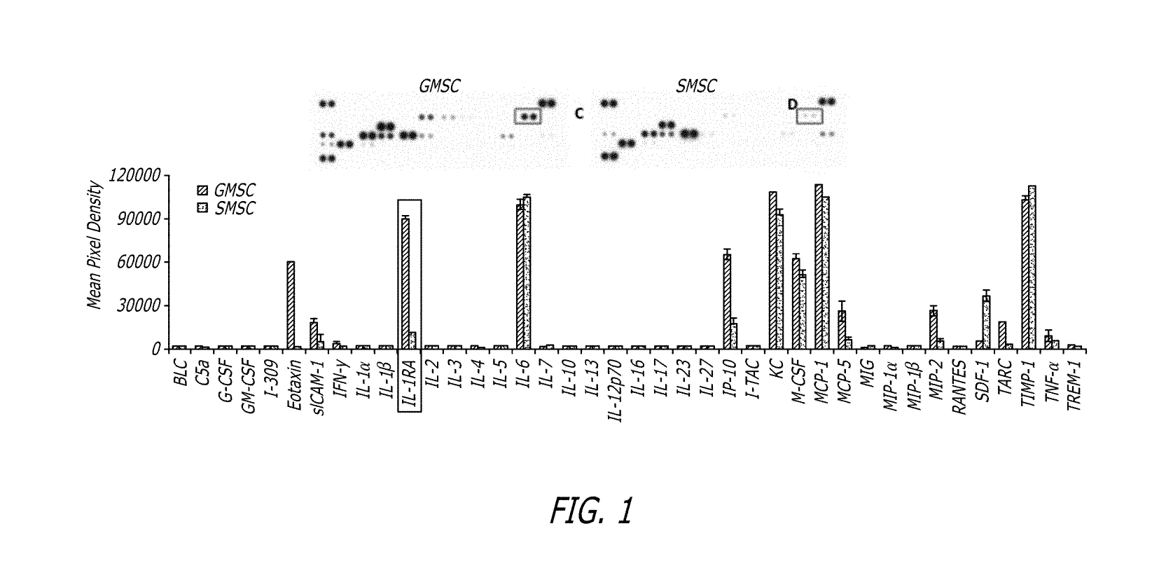

In this example, both gingiva derived mesenchymal stem cells (GMSCs) and skin derived mesenchymal stem cells (SMSCs) were cultured form the same mouse and the culture supernatant was collected for a cytokine array. The cytokines derived from these two types of cells had a significantly different profile (FIG. 1).

Among these differences, one of the anti-inflammatory cytokine, IL-1RA showed a significantly higher expression level in GMSCs when compared with SMSCs. Inflammation, the first stage of wound repair, occurs immediately after tissue damage. Meanwhile, components of the coagulation cascade, inflammatory pathways and immune system are required to prevent ongoing blood losses and infection (Gurtner et al., 2008). A key damaging player in the pro-inflammatory response in numerous and diverse injuries is the cytokine interleukin-1 (IL-1) (Allan et al., 2005). Within the IL-1 ligand family, IL-1RA is a natural receptor antagonist (Dinarello, 2010). IL-1RA binds IL-1R1 with an affinity higher than that of IL-1 but fails to recruit the IL-1RAcP (Garlanda et al., 2013), therefore, inhibit the downstream pathway being activated.

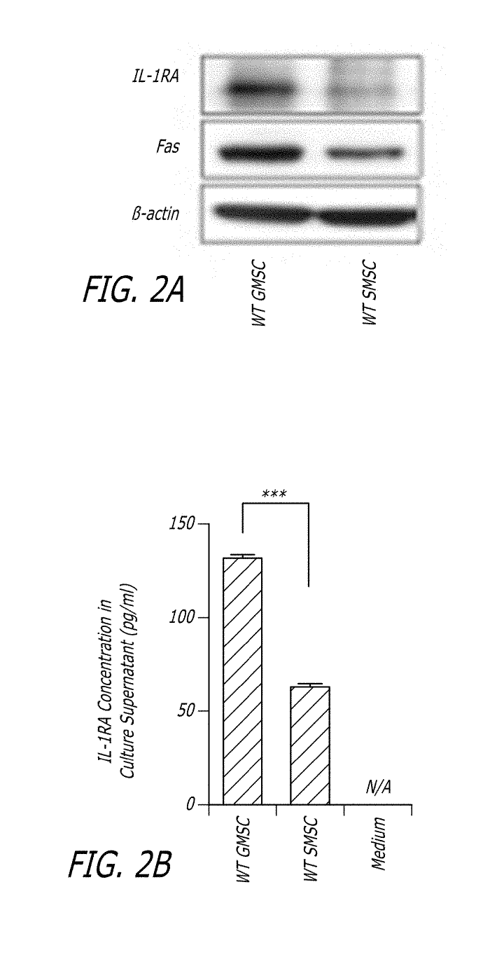

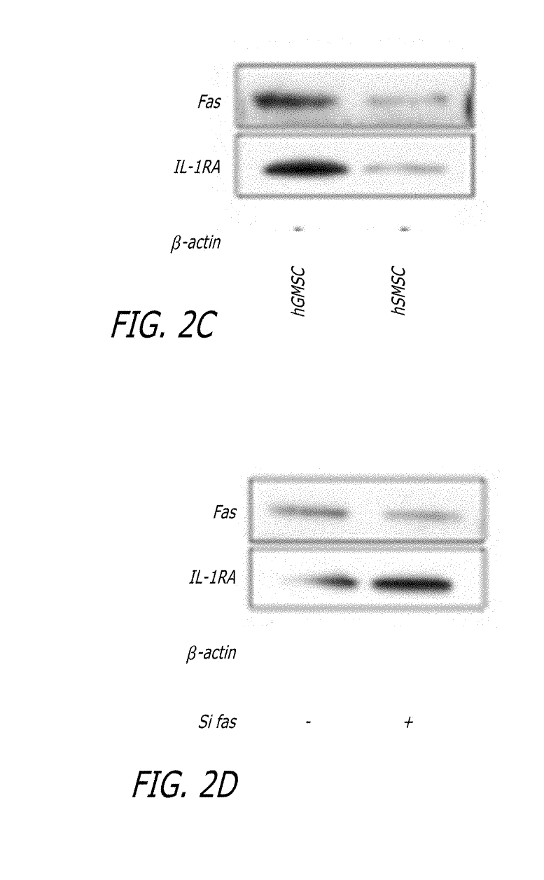

The western blotting assay was used to show that SMSCs express less IL-1RA when compared with GMSCs in wild-type control mice (FIG. 2A), while the enzyme-linked immunosorbent assay (ELISA) result showed that GMSCs also released higher level of IL-1RA in culture supernatant compared with SMSCs (FIG. 2B).

This finding was also tested on the human cells. Human GMSCs also showed an elevated expression of IL-1RA both in cells and in culture supernatant when compared with the human SMSCs (FIG. 2C). This finding was also confirmed on human GMSCs. When fas siRNA was used to knock down the FAS expression on human GMSCs, the IL-RA expression level in cell increased dramatically (FIG. 2D).

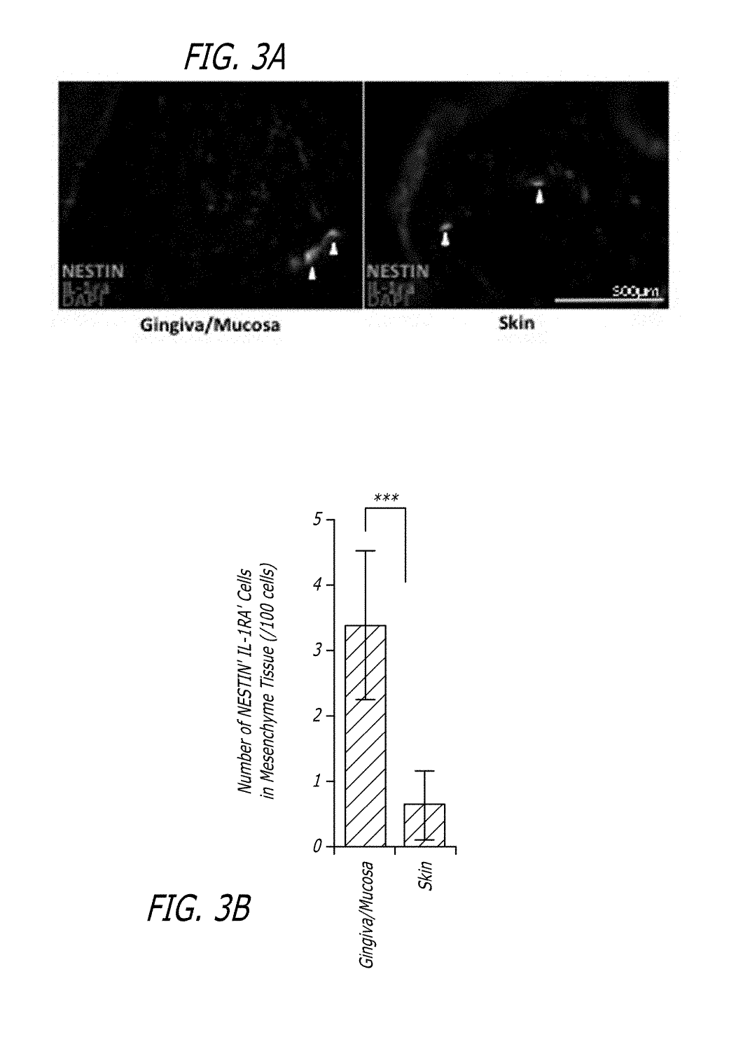

To further confirm the IL-1RA expression profile in in vivo scenario, the NESTIN was used as a marker to label the MSCs as previously described (Méndez-Ferrer et al., 2010) to localize the MSCs in vivo. The immunofluorescence staining showed NESTIN⁺ mesenchymal stem cells were positive with IL-1RA expression in gingiva while rarely observed in skin (FIG. 3A). Statistical analysis showed that in gingiva/mucosa, there are significantly higher number of IL-1RA⁺ and NESTIN⁺ mesenchymal stem cells when compared with skin (FIG. 3B).

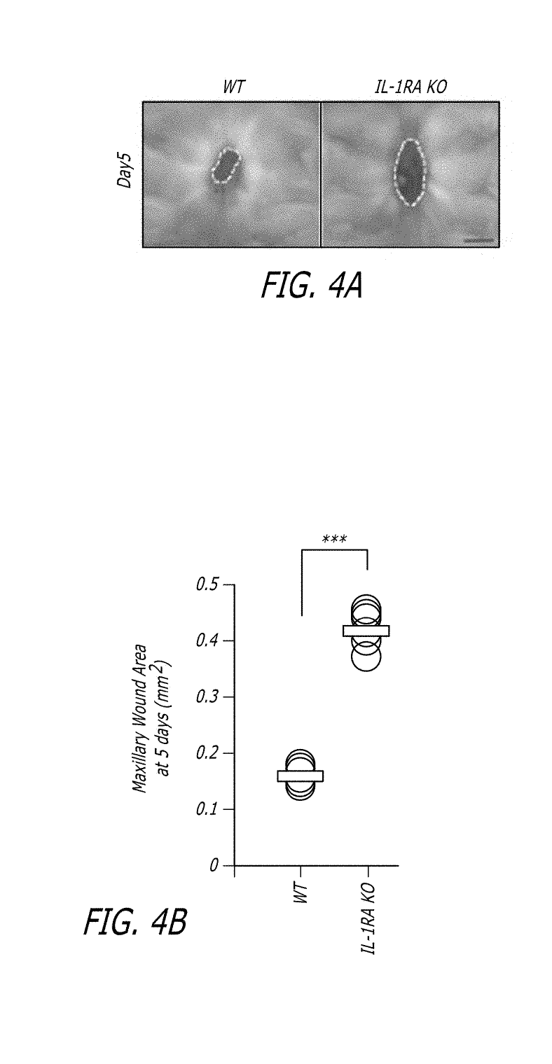

In order to confirm the contribution of IL-1RA in the GMSCs to gingiva mucosa wound healing, the mucosa wound on IL-1RA KO mouse and WT mouse were compared. An about 1.5 mm in diameter of skin was punched on the palate of the mice and the mice was fed with the soft food. The mice were sacrificed after a predetermined time and the wound area was calculated for statistical analyses. The data showed that wound healing was significantly delayed in IL-1RA KO mice at each time point (about 5 days after the wound was formed) (FIG. 4A). Statistical analysis confirmed what histological observation indicated (FIG. 4B).

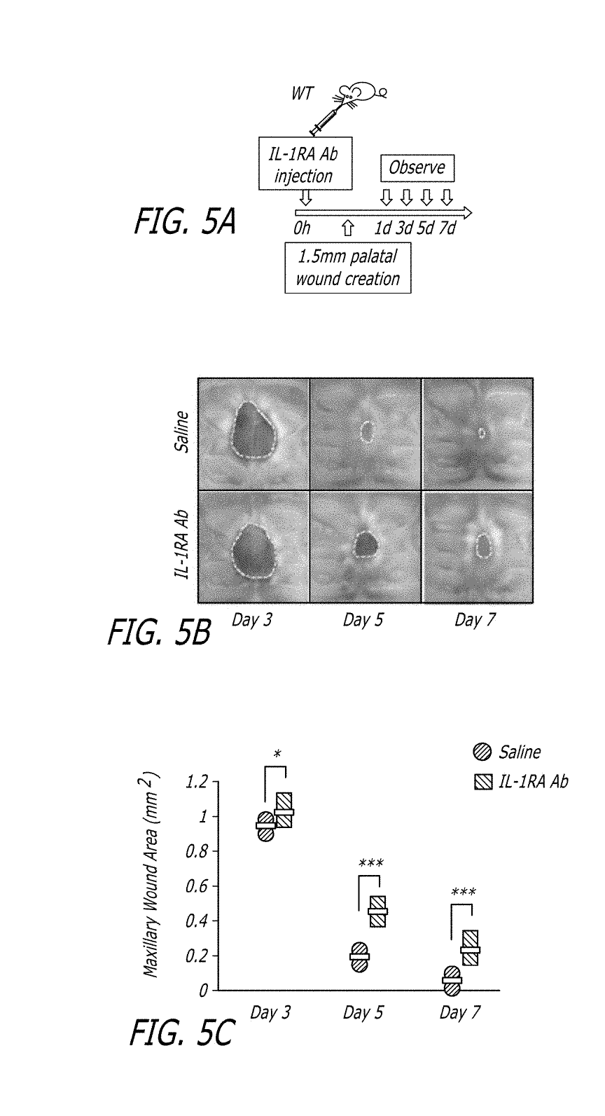

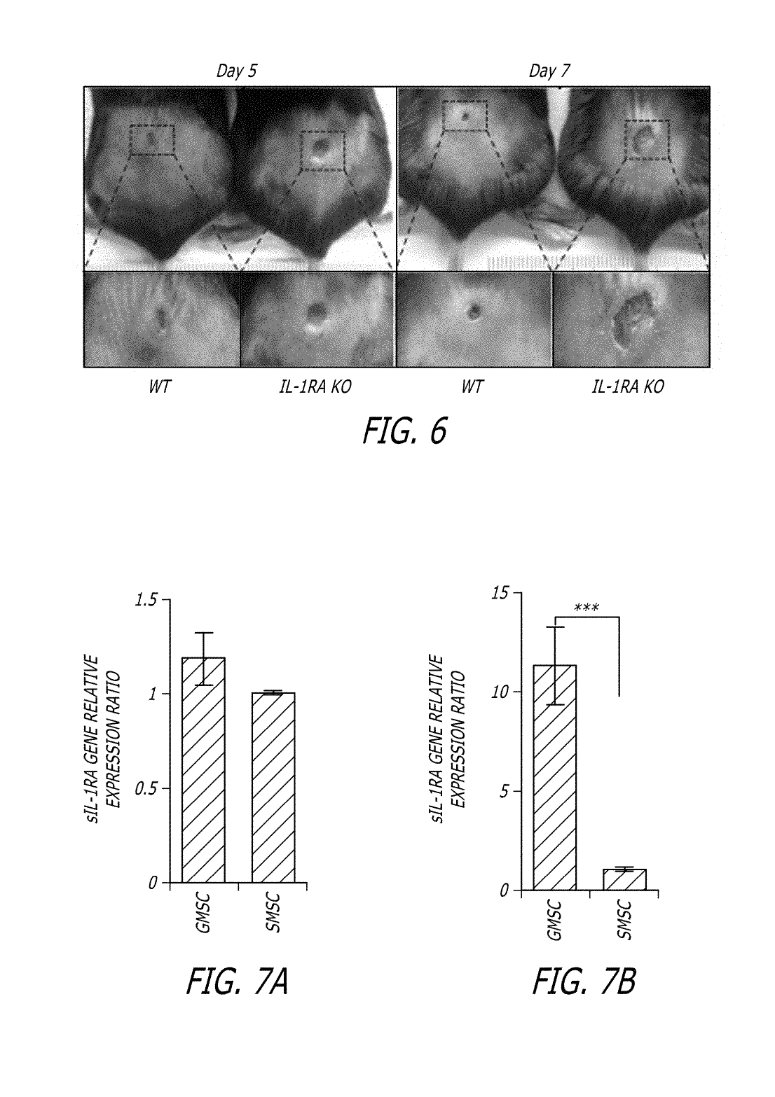

In order to exclude the effect of the IL-1RA secreted by other types of immune cells in a wound at a later stage, the IL-1RA neutralizing antibody was also used to block the IL-1RA in wild-type mice before the wound formation (FIG. 5A). The results were consistent with those shown with the IL-1RA KO mouse, which suggests that the blockage of the IL-1RA in the beginning stage of the wound was enough to delay the wound healing in mucosa (FIG. 5B). Statistical analysis showed that during each time course, the IL-1RA neutralizing antibody significantly delayed the wound healing process in wild type mice (FIG. 5C). It was also confirmed that IL-1RA knockout mice showed delayed skin wound healing (FIG. 6).

### Example 2. FAS May Serve as a Key Molecule in the Secretion of IL-1RA

There are four isotypes of IL-1RA found up till now. One of the isotype is a secreted soluble form of IL-1RA with a signaling peptide at the beginning of the protein, which can actively release from cells. In addition to secreted soluble IL-1RA, there are also intracellular isoforms, which are usually considered a reservoir of IL-1RA, to be released upon cell death, limiting the proinflammatory action of tissue damage (Muzio et al., 1995).

The real-time PCR data showed that GMSCs and SMSCs had same mRNA expression level of soluble IL-1RA (FIG. 7A), while GMSCs had a significantly higher mRNA expression level of intracellular IL-1RA when compared with SMSCs (FIG. 7B).

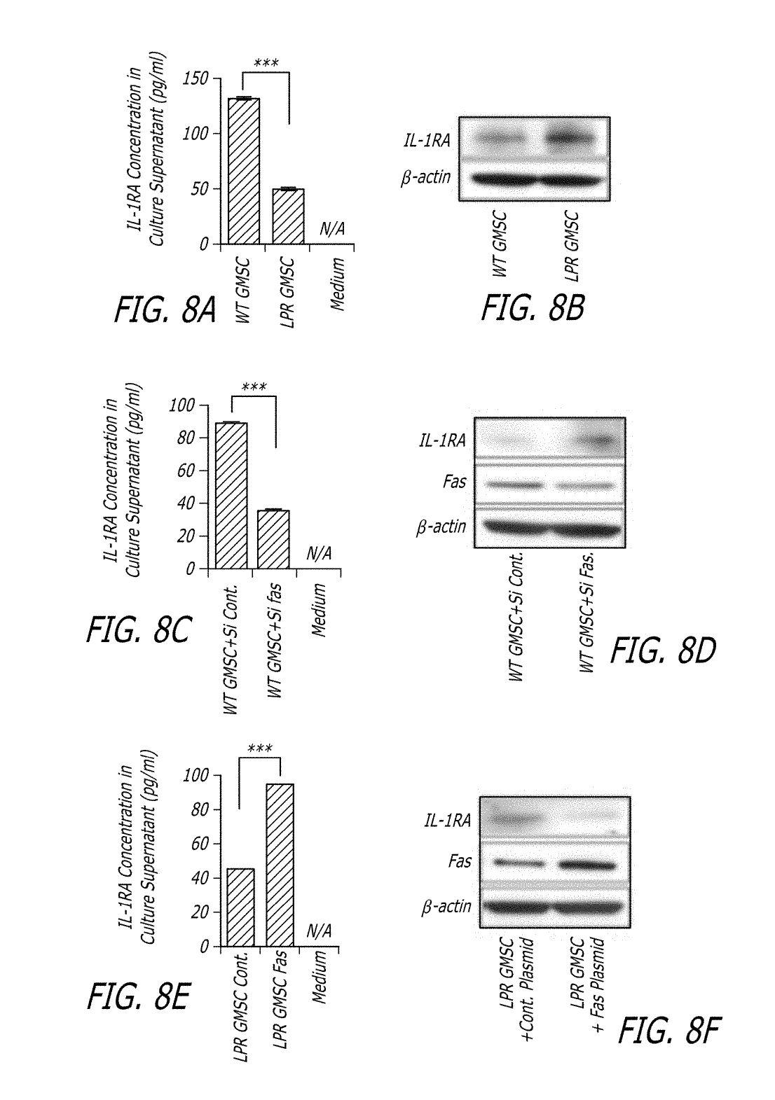

As shown previously that fas KO (Ipr) mice may have deficiency in cytokine release on bone marrow mesenchymal stem cells (BMMSCs). Therefore, two cell types, wild-type GMSCs (WT) and Fas deficient GMSCs (Ipr) were tested for their cytokine release profile. It was shown that WT GMSCs secreted a higher level of IL-1RA in culture supernatant than Ipr GMSCs by ELISA (FIG. 8A). Also, the western blotting assay showed that Ipr GMSCs remained more IL-1RA in cytoplasm (FIG. 8B). To further verify this finding, when the WT GMSCs were treated with fas siRNA, the IL-1RA protein was blocked in the cell with less secreted into the culture supernatant shown by ELISA (FIG. 8C) and with more remained in cell as was shown by western blotting assay (FIG. 8D). Moreover, when FAS was over expressed in Ipr GMSCs, the IL-1RA secretion was enhanced as more IL-1RA was detected in the culture supernatant by ELISA (FIG. 8E) and less IL-1RA remained in cells as shown by western blotting assay (FIG. 8F).

### Example 3. FAS May Work Together with CAVEOLIN1 and SNARE Family Member in IL-1RA Releasing

In this example, the cell membrane transportation was investigated. Since the type of intracellular IL-1RA does not have the signaling peptide allowing it to be automatically transported outside of the cell, it may be packaged into vesicle and transported by proper machinery system on the cell membrane. Therefore, first, a main lipid raft structure named caveolae was found. It dynamically participates in a number of cellular processes including signal transduction, lipid regulation and membrane trafficking (Parton et al., 2013), which has a key protein CAVEOLIN 1 (CAV1). Besides, the Soluble NSF Attachment Protein Receptor (SNARE) family proteins may also be involved. SNARE proteins are a large protein superfamily to mediate the exocytosis of cellular transport vesicles with the cell membrane in yeast and mammalian cells (Sutton et al., 1998). Some key molecules in this family were SYNTAXIN, SNAP25 and Vesicle Associated Membrane Protein (VAMP).

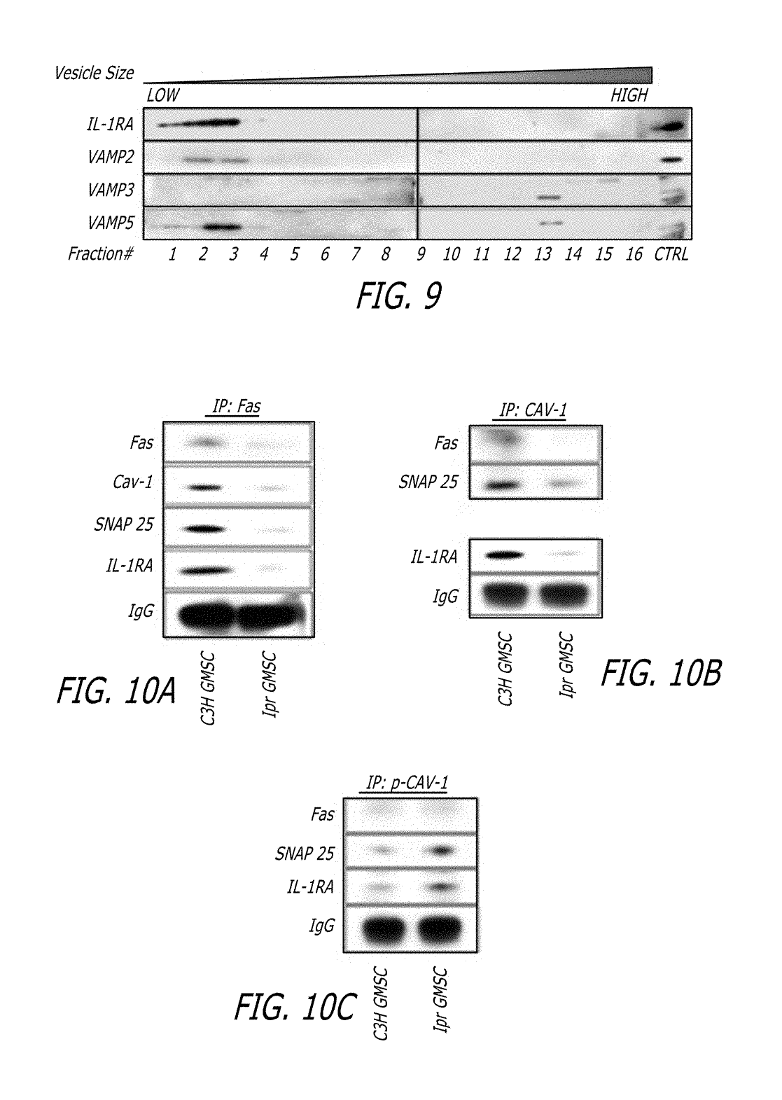

These key molecules were tested to see any of them work together with FAS to mediate the IL-1RA release. First, the sucrose gradient centrifuge methods were used to determine whether IL-1RA was packaged within certain size of the vesicles. As show in FIG. 9, IL-1RA appeared in fractions #1-#4, which were the smallest sized fractions. It was found that the Vesicle Associated Membrane Protein 5 (VAMP5) was co-localized with IL-1RA in same fractions. The immunoprecipitation experiment was carried out to confirm the molecules functioning in complex for the IL-1RA containing vesicle releasing. The western blotting assay of the protein pulled down by FAS antibody showed that in WT GMSCs, FAS were binding with CAV1, SYNTAXIN, SNAP25, VAMP5 and IL-1RA (FIG. 10A-10C).

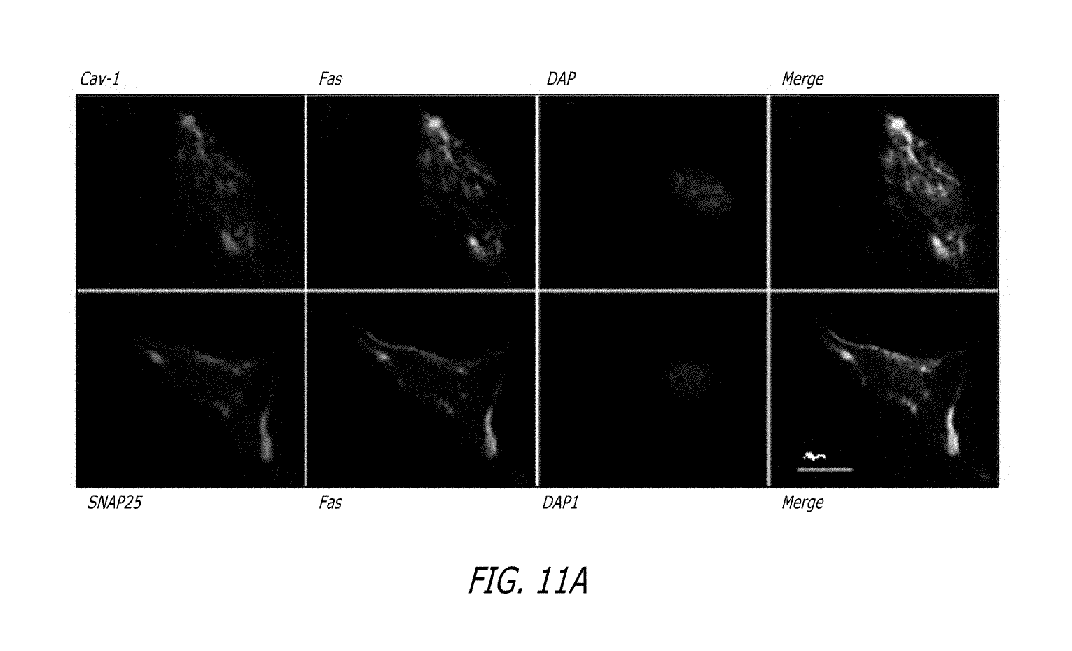

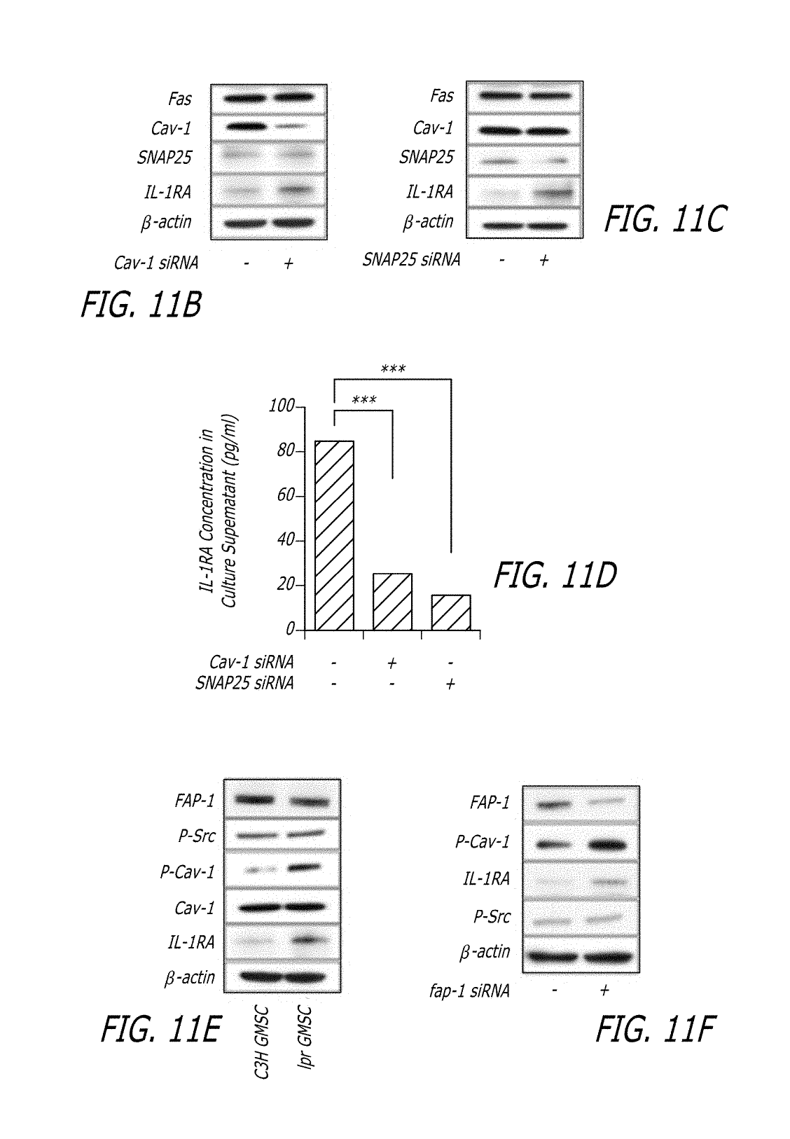

The binding was also confirmed at the cell level, as the immunofluorescence staining showed co-localization of FAS, CAV-1, SYNTAXIN and SNAP25 on WT GMSCs membrane (FIG. 11A-11D). To further verify the complex components, an immunoprecipitation experiment was carried out to pull down every of the previously detected molecule to investigate their binding details. As shown by western blotting assay, when the protein was pulled down by SYNTAXIN antibody, FAS, CAV1, SNAP25, VAMP5 and IL-1RA were found to bind together in WT GMSCs while not in Ipr GMSCs (FIG. 11E, 11F). However, when the CAV-1 antibody was used to pull down the target protein, it was found that all the other molecules remained the same trend as seen in the previous immunoprecipitation except SYNTAXIN, which showed a similar binding in both WT and Ipr GMSCs (FIG. 10B). The result indicated that in WT and Ipr GMSCs, the CAV1 can bind with the SYNTAXIN indifferently.

The CAV1 protein can be phosphorylated at Tyr14 and this phosphorylation has been linked to membrane internalization, endocytosis, focal adhesion stability and dynamics, directional cell migration and extra cellular matrix remodeling (Parton et al., 2013 and del Pozo et al., 2005). Therefore, the phospho-CAV1 expression was tested in both WT and Ipr GMSCs. It was found that there is a much higher expression of the phospho-CAV1 in Ipr GMSCs as shown by western blotting assay (FIG. 11E).

In previous studies, CAV1 was usually believed to be phosphorylated by Src family proteins. However, when the universal phospho-Src expression was tested in both WT and Ipr GMSCs, there was no difference (FIG. 11E). Since the Ipr GMSCs have the mutation on fas gene, FAS may be the key mediator in this phosphorylation. FAS related phosphatase was searched and found FAS Associated Phosphatase-1 (FAP-1) may function in CAV1 dephosphorylation. Though the expression of the FAP-1 in both WT and Ipr GMSCs remained the same (FIG. 11E), the siRNA was used to knock down the fap-1 gene expression to verify its function in cell. When the FAP-1 expression was knocked down in WT GMSCs, the phospho-CAV1 expression was increased but the phospho-Src remained unchanged. Consequently, the IL-1RA concentration in cytoplasm was increased (FIG. 11F). The immunocytofluorescence staining was also used to confirm the phospho-CAV1 expression on the cell level.

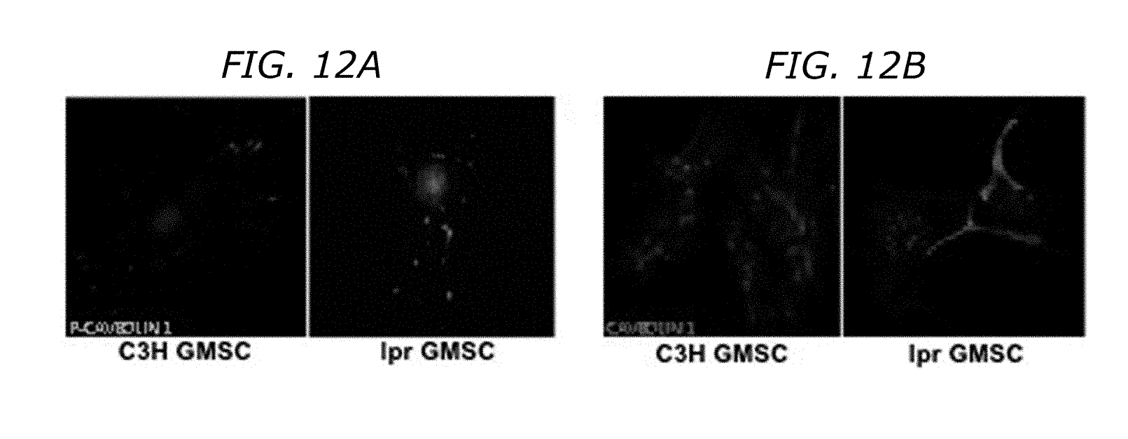

As shown by the immunofluorescence staining, Ipr GMSCs expressed higher level of phospho-CAVEOLIN-1 (FIG. 12A). Previous studies showed that the CAV1 phosphorylation may cause the alteration of the caveolae structure in order to fulfill the physiological functions. The immunofluorescence staining also showed the altered the caveolae distribution in Ipr GMSCs (FIG. 12B). In this example, the western blotting assay was used to analyze the proteins by pulling down by phospho-CAV1 immunoprecipitation. FAS, SYNTAXIN, IL-1RA and VAMP5 were rarely found bound with the phospho-CAV1 in both WT and Ipr GMSCs. However, more SNAP25 was bound with phospho-CAV1 in Ipr GMSCs when compared with WT GMSCs (FIG. 10C). Thus, these results showed that the phosphorylation of CAV1 would bind with the SNAP25, therefore, inhibiting the SNAP25 binding together with SYNTAXIN to trigger the machinery complex to release the IL-1RA containing vesicles.

To further confirm this complex on the functional level, the siRNA was used to knock down cavi, syntaxin, snap25 and vamp5 gene expression in WT GMSCs. The western blotting assay showed that knocked down protein expression of the CAV1, SYNTAXIN, SNAP25 and VAMP25 all leaded to the increase of the IL-1RA expression in cells (FIG. 11B). And these were also confirmed by ELISA assay, which showed a decrease of the IL-1RA concentration in cell culture supernatant (FIG. 11D).

### Example 4. IFN-γ Signaling May Modulate the IL-1RA Containing Vesicle Release

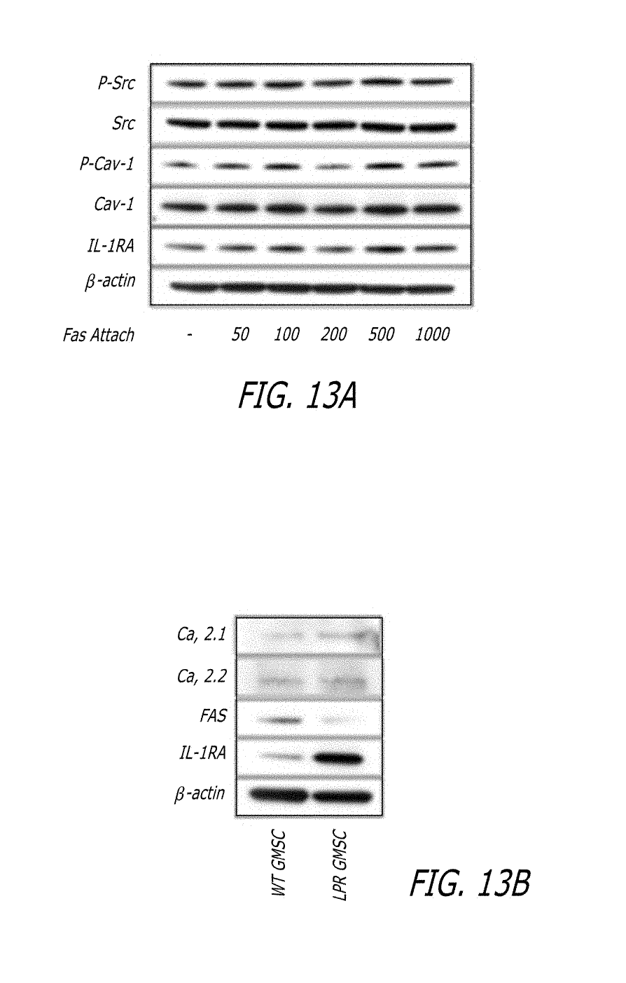

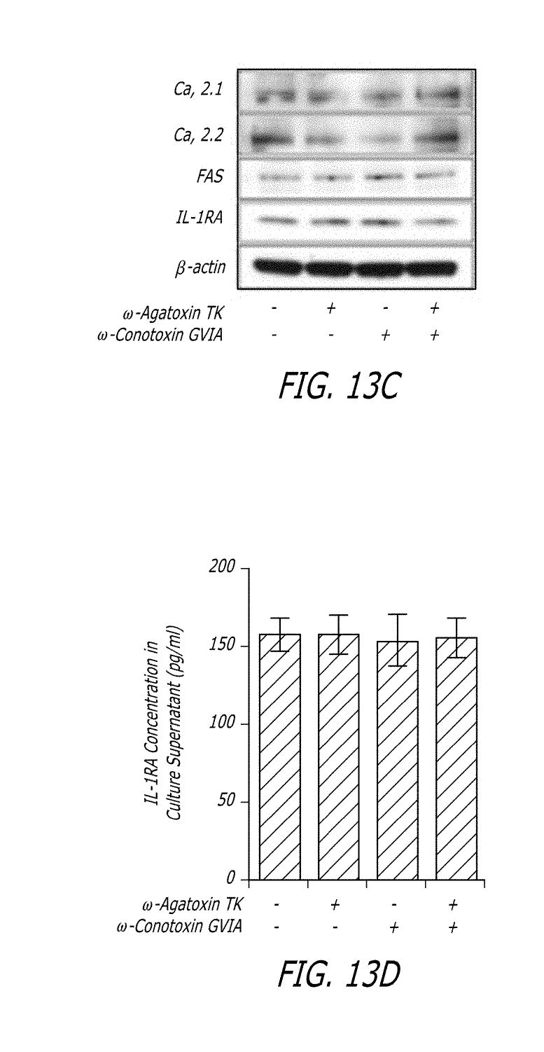

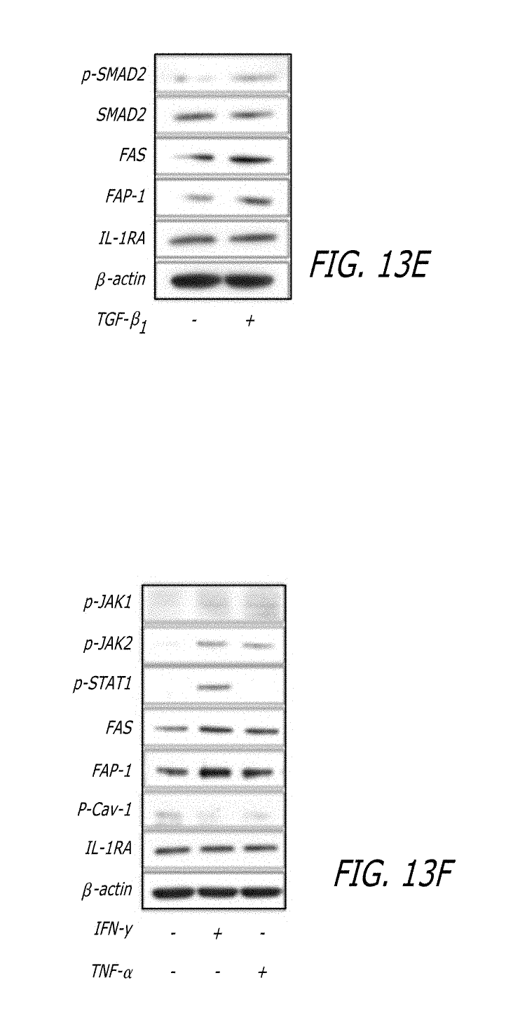

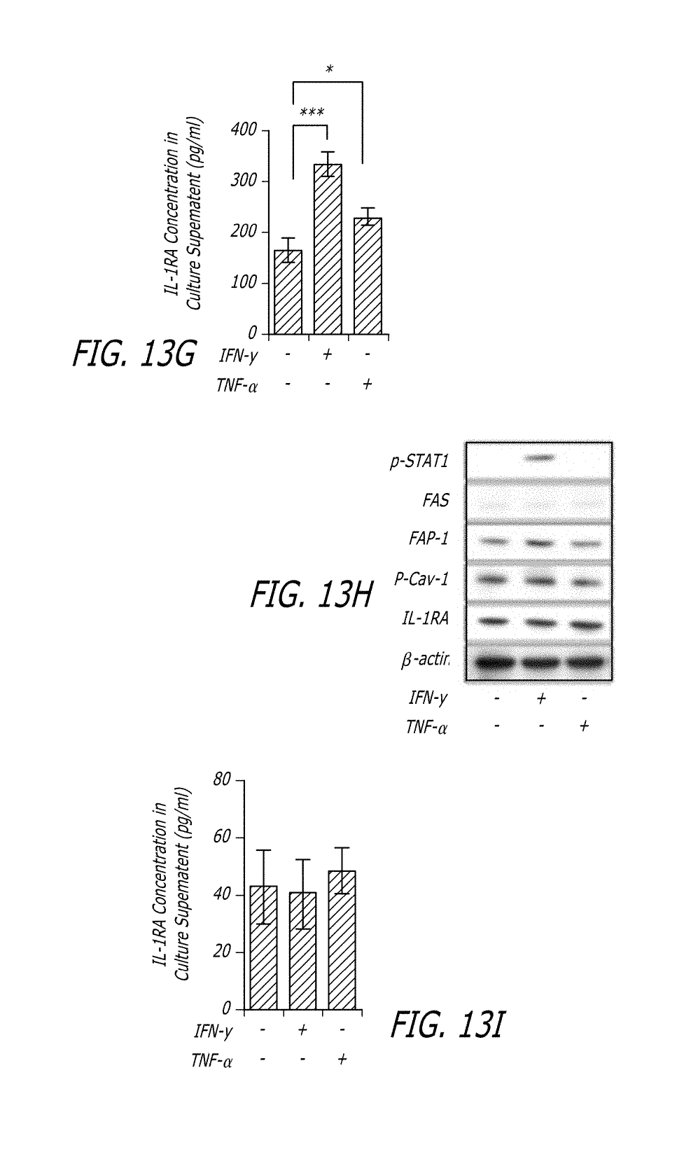

The release mechanism of pathophysiological microenvironment was investigated in this example. First, regulation of the IL-1RA releasing through FAS signaling was tested. The WT GMSCs were tested with different concentration of the FAS activating antibody to activate the FAS signaling. There was no change of the phospho-CAV1, phospho-Src and IL-1RA expression level in cells as shown by western blotting assay (FIG. 13A).

Since the SNARE family members, which were involved in microvesicle release, were being activated by calcium influx in neuron system, and then, calcium influx control of the IL-1RA release was tested. Western blotting assay confirmed that two major calcium channel structure proteins functioning in neuron transmitter release—Caᵥ2.1 and Caᵥ2.2 showed very low expression in WT GMSCs (FIG. 13B). Then, ω-Agatoxin TK and ω-Conotoxin GVIA, the antagonist of these two proteins, were used to block their function in cells to investigate change of the IL-1RA expression levels. Western blotting data showed that the blockage of both Caᵥ2.1 and Caᵥ2.2 did not alter the IL-1RA expression level in cells while the FAS expression level also remained same (FIG. 13C). The ELISA assay was used to confirm the IL-1RA concentration in the cell culture supernatant, which showed no change in any of the antagonist (FIG. 13D).

The environmentally derived signaling may modulate the IL-1RA release. The cell was treated with the TGF-β, which was abundant in wound tissue. It was found that the TGF-β signaling pathway was activated in WT GMSCS as the phospho-SMAD2 expression up-regulated. However, the IL-1RA expression level did not change in cells as shown by western blotting (FIG. 13E). This was also confirmed by ELISA assay, which showed that the IL-1RA concentration remained unchanged in cell culture supernatant after TGF-β treatment (FIG. 13E).

Another important category of signaling was that the inflammatory cytokines emerged after the wound creation. Therefore, the most common inflammatory cytokines IFN-γ and TNF-α were chosen to test their function on IL-1RA release. It was found that when WT GMSCs were treated by IFN-γ and TNF-α, both FAS and FAP-1 expression were up-regulated. Consequently, the phospho-CAV1 level decreased (FIG. 13F). The ELISA assay showed that after the treatment of IFN-γ and TNF-α, the IL-1RA concentration in the cell culture supernatant increased (FIG. 13G). Though TNF-α can also trigger the IL-1RA release in WT GMSCs, when compared with the IFN-γ, its stimulation effect was mild.

### Example 5. IL-1RA Substitute May Ameliorate the Skin Wound Healing

In this example, treatment of skin wounds by the IL-1RA substitute was investigated. An example of IL-1RA substitute may be Kineret® (anakinra), which is a recombinant, nonglycosylated form of the human IL-1RA. Anakinra differs from native human in that it has the addition of a single methionine residue at its amino terminus. It may be produced by recombinant DNA technology using an E coli bacterial expression system. The composition may comprise IL-1RA substitute with a concentration, for example, in the range of 1 mg to 1,000 mg per mL of the composition; or 10 mg to 200 mg per mL of the composition. The composition may comprise IL-1RA substitute with a concentration, for example, in the range of 1 mg to 1,000 mg per gram of the composition; or 10 mg to 200 mg per gram of the composition.

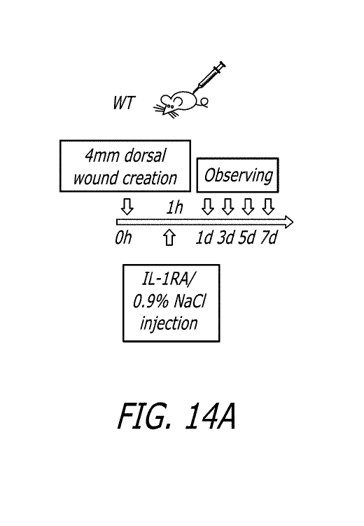

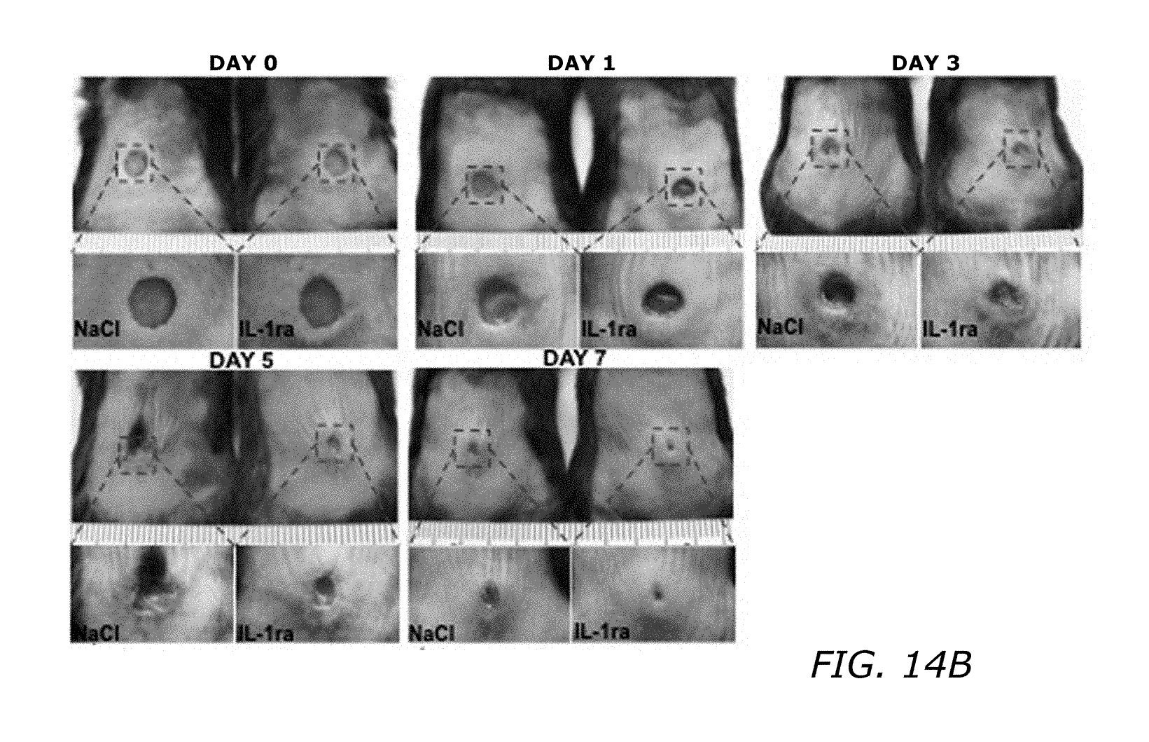

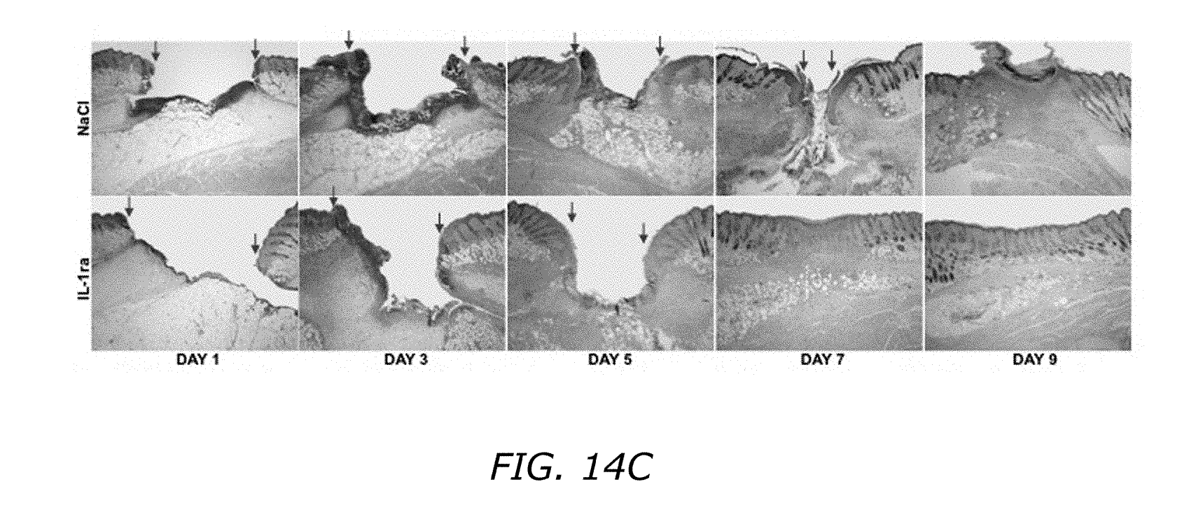

Wounds on dorsal skin of the mice were formed by using a 4 mm diameter punch and the IL-1RA substitute was infused around the wound site about 1 hour after the wound formation. The samples were collected and analyzed at different time points (FIG. 14A). As the data showed, the skin wounds healed much faster in the IL-1RA treated group when compared with the saline treated control at each time point (FIG. 14B).

The statistical analysis of the results indicated the IL-1RA substitute significantly improved the wound healing process in WT mice (FIG. 14B). The samples were further analyzed by histological sectioning using hemotoxylin and Eosin (H&E) staining. The results showed less inflammatory cells infiltration and better collagen deposition in the IL-1RA treatment group (FIG. 14C).

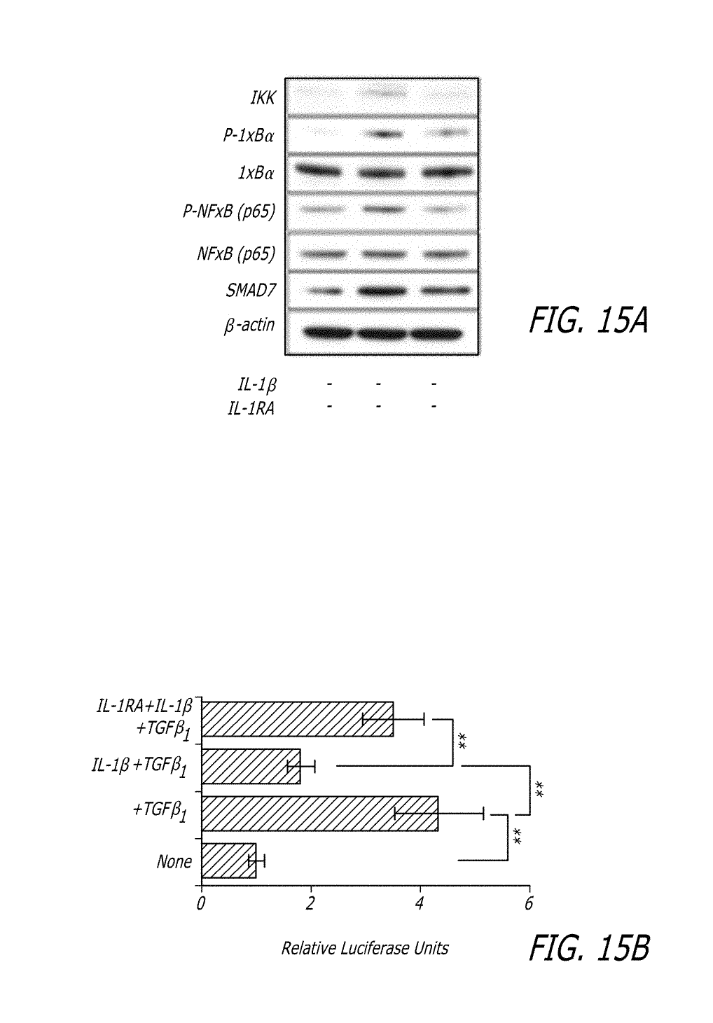

The skin fibroblast method was used to investigate contribution of IL-1RA to the wound healing. The skin fibroblast was treated with IL-1β or IL-1β together with IL-1RA. The western blotting assay showed that the skin fibroblast, when treated with IL-1β, had an elevated NF-κB signaling pathway leading to SMAD7 production, which inhibited the TGF-β pathway, while the administration of IL-1RA can antagonize the IL-1β function (FIG. 15A). Also, the TGF-β reaction reporter plasmid was transfected into the skin fibroblast to confirm this finding. The luciferase assay of TGF-β₁ signaling activity showed that the IL-1β largely inhibited the TGF-β₁ effect on the skin fibroblast. However, IL-1RA significantly rescued the inhibition (FIG. 15B).

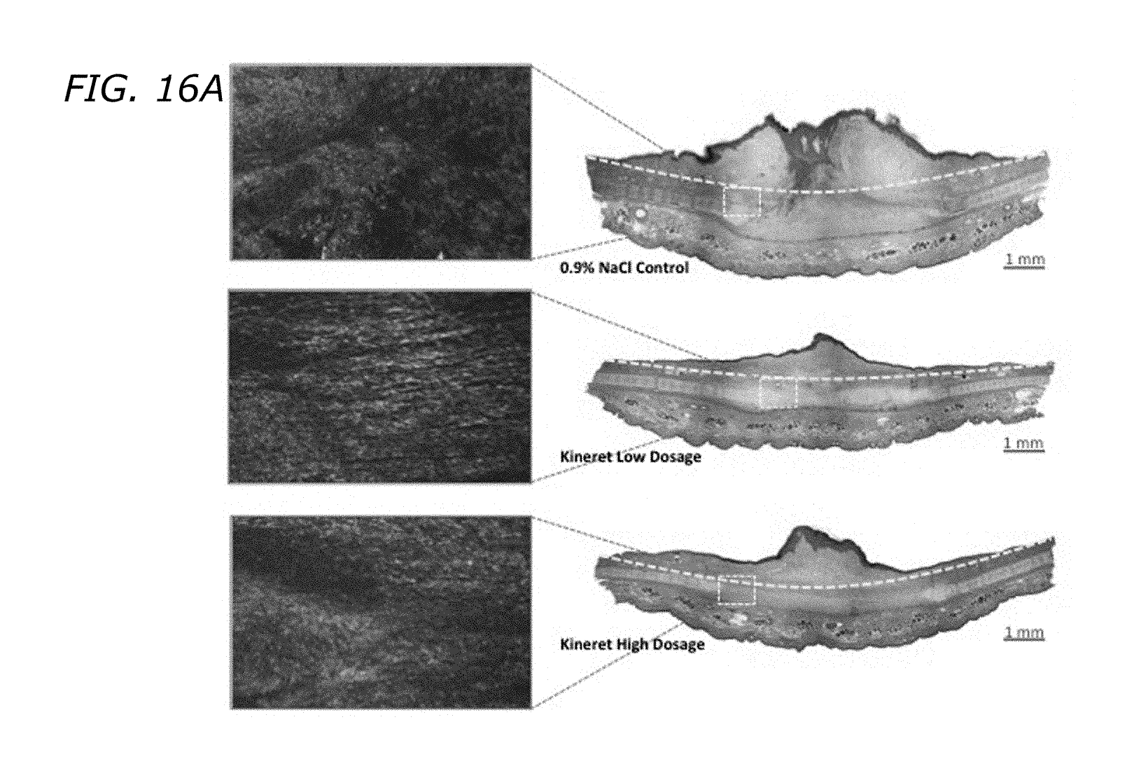

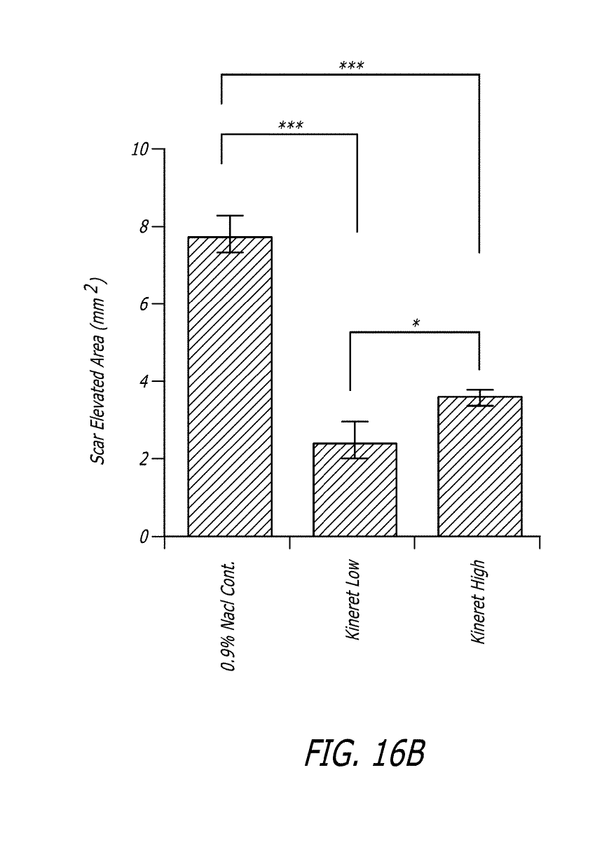

The histological section of the wound sites on both WT and IL-1RA KO mice were also compared. The IL-1RA function on the rabbit ear scar model was also tested. About 6 mm wound was punched down to the cartilage on ventral side of the rabbit ear. A predetermined concentration of the Kineret® (anakinra) was administrated around the wound site about 1 hour after wound creation as was done in dorsal skin. Intriguingly, less scar area and even better aligned collagen deposition were observed in the group of rabbit treated with low dosage of IL-1RA. Although high dosage of IL-1RA treatment slightly compromised this effect, it still showed significantly better treatment effect when compared with the saline treated control group (FIG. 16A). The statistical analysis confirmed that the IL-1RA substitute significantly reduced the scar formation in rabbit ear model (FIG. 16B).

### Example 6

In this disclosure, role of gingiva mesenchymal stem cells (GMSCs) in tissue wound repair was investigated. GMSCs may be stimulated by the adjacent inflammatory environment to release increasing amount of IL-1RA to counterbalance the exaggerated pro-inflammatory response and minimize the side effects of inflammation during the wound healing.

This function was mediated by FAS receptor, which was known for long time as a death-signaling molecule. By association of FAP-1, FAS maintained the key cell membrane trafficking structure caveolae in an un-phosphorylated status to function properly with SNARE family in controlling the IL-1RA containing vesicle release. Notably, this mechanism may not only affect the IL-1RA but also various other cytokines as the cytokine array data showed a complete different profile for the FAS knockout cells. These results suggested that the IL-1RA drug may be used in improving cutaneous wound healing and reducing the scar formation.

MSCs are multipotent cells, which can differentiate into different cell types, such as chondrocytes, osteoblasts, and adipocytes (Pittenger et al., 1999). Recent studies have also suggested that MSCs may influence immune and inflammatory responses (Uccelli et al., 2008). There may be a small number of dormant stem cells residing in almost all tissues that can be activated or migrate to sites of damaged tissue contributing to its repair (Phinney et al., 2007).

Since MSCs interact closely with various stromal cells and inflammatory cells on the site of damage by their immunosuppressive character (Sasaki et al., 2008 and Wu et al., 2007), MSC-derived trophic factors play an important role in these cell-to-cell interplays. In animal models, MSC-conditioned medium could, to some extent, mimic the therapeutic effects of MSCs in enhancing wound healing, and improving cardiac function following myocardial infarction (Chen et al., 2008; Gnecchi et al., 2005 and Timmers et al., 2011).

In this disclosure, GMSCs had strong icIL-1RA expression in ground status. These natural antagonists can be released and contribute to the mild Interleukin-1 (IL-1) based inflammatory response in the gingiva/mucosa tissue, resulting in healing of their wounds. The IL-1RA knockout mice showed a significantly delayed wound healing. When IL-1RA substitutes were applied to the cutaneous wound, they also improved the wound healing process. On a rabbit ear scar model, the scar formation was reduced when the IL-1RA applied shortly after the wound creation. These findings showed that gingiva/mucosa had a much-regulated inflammatory response during injury, and the regulatory effect largely depends on the GMSCs' innate function of releasing the anti-inflammatory cytokines.

Tissue wound usually initiates the activation of immune and inflammatory cascade. Acute tissue damage is usually followed by immediate inflammation (Medzhitov et al., 2008). Cellular components released from necrotic cells and microvasculature damage lead to enhanced vasopermeability and infiltration of immune cells, which are the main source of inflammatory cytokines (Luster et al., 2005 and Eming et al., 2007). The initial studies on the immunomodulatory effects of MSCs produced conflicting data (Uccelli et al., 2008), suggesting that the immunosuppressive property of MSCs could be affected by specific damage/disease-related tissue microenvironments.

Once MSCs have encountered the microenvironment of injured tissues, many factors, including inflammatory cytokines such as IFN-γ, TNF-α, IL-1, toxins of infectious agents and hypoxia can stimulate them to release various growth factors, including transforming growth factor-b (TGF-β), epidermal growth factor (EGF), fibroblast growth factor (FGF), platelet-derived growth factor (PDGF), vascular endothelial growth factor (VEGF), keratinocyte growth factor (KGF), hepatocyte growth factor (HGF) and insulin growth factor-1 (IGF-1) (Shi et al., 2012; Aguilar et al., 2009 and Hung et al., 2012).

The multitude of paracrine factors may provoke tissue-resident cells such as fibroblasts to promote wound healing through their secretion of extracellular matrix and matrix metalloproteinase. Several studies indicated that the priming of the MSCs by inflammatory cytokines is essential for MSC-mediated immunosuppression. Other studies also showed that pretreatment with inflammatory cytokines can enhance the therapeutic effect of MSCs (Krampera et al., 2006; Sheng et al., 2008 and Mougiakakos et al., 2011).

These data indicated the importance of the local inflammatory condition in regulating the plasticity of MSCs. Up to date, little was known about how these inflammatory stimulus function on MSCs. The limited reports on the mechanism of this type of MSCs “licensing” showing it was in a nuclear factor (NF)-κB-dependent manner.

In this disclosure, it was shown that upon the challenge by the IFN-γ, both FAS and its associate phosphatase (FAP-1) were upregulated through JAK/STAT 1 pathway to maintain the important membrane structure caveolae in an unphosphorylated form. This unphosphorylated status helped the SNARE machinery system within the caveolae structure to function properly on the secretion of IL-1RA containing vesicle.

Tissue injury initiates a complex set of events, which finally lead to wound repair. The wound healing process begins with inflammation. Studies using various models have shown that when compared to similar wounds in skin, wound healing in oral mucosa and gingiva is markedly faster, resulting to significantly reduced scar formation.

The reason for the preferential oral mucosal wound healing may in part depend on the milder and shorter inflammatory response in oral mucosa (Glim et al., 2013; Szpaderska et al., 2003 and Mak et al., 2009). Among all the pro-inflammatory factors immediately emerging around the lesion site, interleukin-1 (IL-1) family members are the first of which to be induced and detected (Hübner et al., 1996). IL-1 is a prototypical proinflammatory cytokine that stimulates both local and systemic responses with detrimental effects in skin lesions, inflammatory arthritis, fever and leukocytosis. However, these responses are also essential in the control and resolution of infections (Miller et al., 2006). Some of these effects are indirect, mediated through the induction of downstream cytokines and other inflammatory mediators (Dinarello et al., 1996).

In addition, IL-1 is also involved in the regulation of adaptive immune responses by inducing the differentiation of type 17 T-helper cells and the production of IL-17 in mice and humans (Chung et al., 2009 and Acosta-Rodriguez et al., 2007). The proinflammatory activities of IL-1 are strictly controlled by several endogenous inhibitors, such as the first described naturally occurring antagonist IL-1RA.

IL-1RA binds tightly to IL-1R1, thereby blocking binding of IL-1α and IL-1β. In the presence of IL-1RA, the reduction of IL-17A and IL-22 is 82% and 71%, respectively (van de Veerdonk et al., 2012). Moreover, unlike IL-1α and IL-1β, receptor-bound IL-1RA does not allow recruitment of IL-1RAP (Greenfeder et al., 1995), thus protect the downstream signaling being activated.

It was shown that the balance between IL-1 and IL-1RA determines the overall inflammatory response (Arend, 2002). Deficiency of IL-1RA in mice results in spontaneous and lethal arteritis, destructive arthritis, and psoriatic-like skin lesions, as well as increased susceptibility to carcinogenesis (Horai et al., 2000 and Nicklin et al., 2000).

Children born with a genetic deficiency of IL-1RA or functional inactive IL-1RA suffer from severe systemic and local inflammation, including pustular skin eruptions, vasculitis, osteolytic lesions, and sterile osteomyelitis (Aksentijevich et al., 2009 and Reddy et al., 2009). IL-1RA is produced as four different isoforms by using different first exons and alternative mRNA splicing and translation initiation codons. One isoform is secreted (sIL-1RA), whereas the three others lack a consensus leader peptide and remain intracellular (icIL-1RA1, icIL-1RA2 and icIL-1RA3). Intracellular forms of IL-1RA are believed to release following cell death and act extracellularly on IL-1R1, similarly to the secreted form (Palmer et al., 2007).

In this disclosure, it was found that besides the cell death, proinflammatory cytokines such as IFN-γ can stimulate the GMSCs to release high level of icIL-1RA into the wound area, counterbalance the IL-1 signaling. Therefore, this may partially explain the reason of the mild inflammation observed after gingiva wound injury.

It was also shown that if the GMSCs are blocked on this response, either through the inhibition of the IFN-γ or IL-1RA, gingiva wound healing may be significantly delayed when compared with the WT control. These results comply with the study of skin wound on IL-1RA knockout mice (Ishida et al., 2006), proving that the newly found IL-1RA releasing character on GMSCs is decisive in gingiva wound healing.

FAS (CD95/APO-1) is a member of the tumor necrosis factor (TNF) superfamily (Trauth et al., 1989 and Yonehara et al., 1989). Studies on the FAS system focused primarily on its role in apoptosis. FAS is a prototypical death receptor with an intracellular “death domain” (DD), which is essential for transduction of the apoptotic signal (Locksley et al., 2001). Upon ligation by FAS-Ligand, FAS receptors multimerize and recruit FAS-associated death domain (FADD) through DD-DD interactions (Scott et al., 2009). FADD in turn recruits pro-caspase-8 through death effector domain (DED) forming death inducing signaling complex (DISC) (Tibbetts et al., 2003).

Depending on different signaling activation, apoptosis proceeds through either extrinsic or intrinsic apoptosis pathway (Scaffidi et al., 1998). Recent studies suggest, however, FAS may mediate other cellular non-apoptotic responses such as suppression of T-cell activation (Kovacs et al., 1995), promoting the proliferation of CD3-activated primary T cells (Alderson et al., 1993) and a variety of non-immunological cells including, fibroblasts, tumor cells, hepatic stellate cells and neural cells (Freiberg et al., 1997; Chen et al., 2010; Desbarats et al., 2000; Reinehr et al., 2008 and Lambert et al., 2003).

In addition, FAS system can promote cell migration and invasion, in particular in apoptosis-resistant malignant cells (Barnhart et al., 2004; Li et al., 2009; Trauzold et al., 2005 and Letellier et al., 2010). It was also shown that FAS signaling could trigger inflammation by promoting the inflammatory factors expression in different types of cells (Brint et al., 2013). Such factors may in turn recruit inflammatory cells, exacerbating the inflammatory process. FAS-induced inflammatory factor production predominantly involves the activation of transcription factor NF-κB, mitogen-activated protein kinase (MAPK) pathways (Zhang et al., 2009 and Palao et al., 2006) and the cross talk between toll-like receptor (TLR) family (Ma et al., 2004).

Some studies suggested that one of the key mechanisms of regulating the apoptotic or non-apoptotic pathway was the FAS receptor internalization. During the apoptotic process, DISC has been shown to continue binding with the internalized FAS receptor, while inhibition of FAS receptor internalization engages it to induce pro-survival pathways (Eramo et al., 2004). Caveolae and CAVEOLIN-1 (CAV1) have been known to involve in the internalization of FAS receptor complex, and blockade of CAV1-mediated endocytosis diminishes cell apoptosis (Gajate et al., 2001; Zhu et al., 2009 and Guo et al., 2009). Caveolae, with CAV1 as its principle component, are the main structure of lipid rafts that are distinct cholesterol- and sphingomyelin-rich, flask-shaped invaginations (60-80 nm) of the plasma membrane. It is dynamically participate in a number of cellular processes including signal transduction, lipid regulation and membrane trafficking (Parton et al., 2013).

Phosphorylation of CAV1 at Tyr14 has also been linked to membrane internalization, endocytosis, focal adhesion stability and dynamics, directional cell migration and extra cellular matrix remodeling (Parton et al., 2013 and del Pozo et al., 2005). However, the functional switch of CAV1 between its phosphorylated and un-phosphorylated form is not yet clear.

It was previously found that FAS receptor contributed to cytokine secretion as the FAS mutant Ipr bone marrows mesenchymal stem cells (BMMSCs) showed a significantly altered cytokine releasing profile when compared with the WT control (Akiyama et al., 2012).

In this disclosure, it was shown that adding FAS activating antibody did not influence the IL-1RA releasing of GMSCs, which suggested that non-apoptotic FAS pathway played an anchoring role in cytokine releasing. When under the inflammatory environment, FAP-1 could be induced and function together with FAS receptor to maintain the CAV-1 in un-phosphorylated form, which thereby inhibit the FAS mediated death pathway being activated through internalization, meanwhile keeping the SNARE complex to be properly functioning in transporting the IL-1RA containing vesicle. These findings to some extent explained the mechanism of how MSCs can escape from the immediate elimination by the innate immune system when infused into circulation for cell therapy.

The discovery that MSCs can modulate the inflammation as well as the host immune system has revolutionized the stem cell therapy. Although most of current investigations focus on the immunomodulatory capability of MSC populations expanded in vitro, the physiological roles of tissue-resident MSCs in immunomodulation have yet be elucidated (Wang et al., 2014). Understanding the function of the tissue-resident MSCs will undoubtedly bring new insight into these unique cells in various pathophysiological conditions. The intercellular communication is generally achieved by direct cell-to-cell contact, secreted paracrine molecules or by the exchange of the extracellular vesicles.

In this disclosure, it was shown that the in situ GMSCs were stimulated by the wound inflammation to secrete more IL-1RA containing vesicles to balance the inflammatory environment. IL-1RA may not be the only peptide, which is involved in the complicated wound healing process.

A wealth of studies showed that vesicles may also contain genetic information such as DNA, mRNA and miRNAs (Valadi et al., 2007). Therefore the microvesicle release system on GMSCs, disclosed here, may also apply to various other containing contents.

Investigations in the past few years have provided solid evidence for the function of MSCs in the treatment of immunological disorders. However, there are still important concerns about the therapeutic use of these unique cells such as the origins of the tissues, the use of autologous or allogeneic MSCs, etc. need to be clarified. The understanding of the detailed physiological role of MSCs in the in vivo scenario will definitely do help to guide the appropriate application of MSCs and optimize to reach better modulation of inflammatory responses at different stages of disease progression.

### Example 7. Methods

The methods disclosed below were used in above examples.

Animals.

Female C57BL/6J (stock 000664), C3H/HeJ (stock 000659), C3.MRL-Fas^{Ipr}/J (stock 000480) and B6.129S7-Ifng^{tm1Ts}/J (stock 002287) mice were purchased from Jackson Lab. All animal experiments were performed under the institutionally approved protocols for the use of animal research (University of Southern California protocol number 11141, 11953 and 11327). Mice 2-3 months of age were used in the experiments.

Antibodies and Reagents

Anti-runt-related transcription factor 2 (RUNX2) and -Osteocalcin (OCN) antibodies were purchased from Millipore (Billerica, Mass., USA). Anti-alkaline phosphatase (ALP), -Nestin, -β-TUBULIN III, -Collagen II, -Sox9 and -Neurofilament Medium (NF-09) antibodies were purchased from Abcam (Cambridge, Mass., USA). Anti-Sca-1-PE, -CD34-PE, -CD44-PE, -CD45-PE, -CD11b-PE, -CD73-PE, -CD117-PE, -CD4-PerCP, -CD25-APC, -IgG1-PE, -IgG₂ₐ-PE, -IgG_{2b}-PE, -CD3ε and -CD28 antibodies were purchased from BD Bioscience (San Jose, Calif., USA). Anti-Foxp3-PE, -IL17-PE, -CD105-PE and -CD90-PE antibodies were purchased from eBioscience (San Diego, Calif., USA). Purified anti-peroxisome proliferator-activated receptor γ (PPARγ), -lipoprotein lipase (LPL) and -FAS Ligand (FASL) antibodies, as well as secondary antibodies, and, were purchased from Santa Cruz Biosciences (Santa Cruz, Calif., USA). Anti-β actin antibody was purchased from Sigma (St. Louis, Mo., USA). EdU imaging kit was purchased from Invitrogen (Invitrogen, Carlsbad, Calif., USA).

Cell Culture.

Gingiva tissues from mouse mandibular molar region were gently separated, minced, and digested with solution containing about 2 mg/mL collagenase type I (Worthington Biochemical, Freehold, N.J., USA) and about 4 mg/mL dispase II (Roche Diagnostics, Indianapolis, Ind., USA) in phosphate buffered saline (PBS) for about 1 hour at about 37° C. Single-cell suspensions were obtained by passing the cells through a 70-μm strainer (BD Biosciences, Franklin Lakes, N.J., USA). All nucleated cells (ANC) were seeded at 1×10⁶ into 100-mm culture dishes (Corning, N.Y., USA) with α-MEM (Invitrogen, Carlsbad, Calif., USA) supplemented with about 20% FBS, about 2 mM L-glutamine (Invitrogen), 55 μM 2-mercaptoethanol (Invitrogen), about 100 U mL⁻¹ penicillin, and about 100 μg mL⁻¹ streptomycin (Invitrogen), followed by an initial incubation for about 48 hours under about 37° C. at about 5% CO₂. The cultures were washed with PBS twice to eliminate the non-adherent cells. Attached cells were cultured for another 12 days under same condition in the complete medium mentioned above.

Histology.

To assess wound healing progress, skin and maxilla wound samples were fixed in about 4% paraformaldehyde (Sigma-Aldrich) and then decalcified (maxilla samples) with about 5% EDTA (pH 7.4), followed by paraffin embedding. Paraffin sections (about 6 μm) were stained with hematoxylin and eosin (H&E) and analyzed by NIH ImageJ software. To perform Immunohistochemistry staining, the paraffin embedded sections were blocked with serum matched to secondary antibodies, incubated with the IL-1RA or IL4 specific antibodies (Santa Cruz Biotechnology, Inc, 1:400) at about 4° C. for overnight, and then stained using VECTASTAIN UNIVERSAL elite ABC kit and ImmPACT VIP Peroxidase Substrate kit (VECTOR) according to the manufacturer's instruction.

ELISA.

Cell culture medium was collected, and IL-1RA protein levels were analyzed by using mouse ELISA Quantikine® Immunoassay kit (R&D Systems MRA00), according to the manufacturer's instructions.

Real-Time PCR.

Total RNAs were isolated from different GMSCs by means of the TRIzol® Reagent (Life Technologies, Invitrogen). RNA samples (about 1 μg) were reverse-transcribed in a Reverse Transcription system (QIAGEN). Primers used were: (sII-1 ra) forward, 5′-AAATCTGCTGGGGACCCTAC-3′ (SEQ ID NO:1), and reverse, 5′-TCCCAGATTCTGAAGGCTTG-3′ (SEQ ID NO:2); (icII-1ra) forward, 5′-AGACACTGCCTGGGTGCTCCT-3′ (SEQ ID NO:3) and reverse, 5′-GTTTGATATTTGGTCCTTGTAAG-3′ (SEQ ID NO:4) and (GAPDH as an internal control) forward, 5′-GAAGGTGAAGTTCGGAGTC-3′ (SEQ ID NO:5), and reverse, 5′-GAAGATGGTGATGGGATTTC-3′ (SEQ ID NO:6). PCR conditions were: about 95° C. for about 5 min, (about 95° C. for about 10 sec, about 50° C. for about 45 sec)×40 times, and about 90° C. for about 10 sec.

Western Immunoblotting.

Cells were lysed in M-PER mammalian protein extraction reagent (Thermo) with protease and phosphatase inhibitors (Roche), and proteins were quantified using protein concentration assay (Bio-Rad Laboratories). About 20 μg of proteins were separated by SDS-PAGE and transferred to about 0.2 μm nitrocellulose membranes (Millipore). The membranes were blocked with about 5% non-fat dry milk and about 0.1% Tween-20 for about 1 hour, followed by incubation overnight with the primary antibodies diluted in blocking solution according to manufacturer's instructions. Antibodies to mouse FAS were purchased from Abcam, Cell Signaling, Millipore, and Sigma-Aldrich. The membranes were then incubated under room temperature for about 1 h in species related HRP-conjugated secondary antibody (Santa Cruz) diluted at about 1:10,000 in blocking solution. Immunoreactive proteins were detected using SuperSignal® West Pico Chemiluminescent Substrate (Thermo) and BioMax film (Kodak).

RNAi and Chemical Reagent Treatments.

GMSCs (about 0.3×10⁶) were seeded to a 60 mm culture plate and treated with Cav1 siRNA (Santa Cruz), Fas siRNA (Santa Cruz), Snap25 [symbol] siRNA (Santa Cruz) according to the manufacturers' instructions. After transfection, cells were either used for protein extraction for Western immunoblotting or for live imaging. For chemical reagent treatments, serum-starved GMSCs were treated with about 200 ng/mL IFN-γ (R&D Systems), about 50 ng/mL TNF-α (Peprotech), or about 1 μg/mL TGFβ neutralized antibody (R&D systems). For Western immunoblotting, MSCs were cultured under growth medium with treatments for about 24 hours, and protein was extracted by using M-PER mammalian protein extraction reagent.

Immunofluorescent Microscopy.

GMSCs were cultured on 2-well chamber slides (Nunc) (about 1×10³/well) and then fixed with 4% paraformaldehyde. The Chamber slides or the previous prepared histological sections were incubated with primary anti CD73 antibody (about 1:400, BD) and anti IL4Rα (about 1:400, BD) at about 4° C. for overnight. And then treated with Alexafluoro 568 or Alexafluoro 488 conjugated secondary antibody (about 1:200, Invitrogen) for about 1 hour at room temperature. Finally, slides were mounted with Vectashield mounting medium (Vector Laboratories).

Luciferase Reporter Assay.

Tgfβ reaction luciferase promoter reporter constructs were purchased from Addgene (plasmid 11767, Wrana et al., 1992). GMSCs cultured in 6-well plates were co-transfected with 2 μg of luciferase reporter and 100 ng of Renilla luciferase expression vector to control for transfection efficiency. Forty-eight hours after transfection, cells were lysed in 1× passive lysis buffer, and luciferase activity was measured using the Dual-Glo Luciferase System (Promega) and a luminometer (Turner Biosystems).

II-1 ra-GFP Plasmid Construction.

II-1ra-GFP expression plasmid constructs were generated by PCR using Pfu polymerase and mouse II-1ra cDNA vector (GE LifeScience MMM1013-202766323) as a template. Primers containing upstream MluI and downstream NheI restriction sites were used to generate II-1ra cDNA fragment (forward, 5′-ACGCGTATGGCTTCAGAGGCAGCCTG-3′ (SEQ ID NO:7) and reverse, 5′-GCTAGCTTGGTCTTCTGGAAGTAGA-3′(SEQ ID NO:8)). Restriction digested PCR products were subcloned into pBI-GFP vector (Addgene plasmid 16623, Cahill et al., 1998). All clones were confirmed by sequencing on both strands. GMSCs cultured on 2-well chamber slides (Nunc) (about 1×10³/well) were transfected with about 2 μg of II-1ra-GFP expression plasmid by Lipofectamine® 3000 (Invitrogen). About 48 hours after transfection, cells were kept in UNO CO₂ Microscope Stage Incubator (okolab) for confocal microscope live imaging (Leica).

Statistics.

All experimental group sizes were chosen to ensure adequate statistical power despite the highly variable nature of the studies performed. No animal excluded, and animals were randomly assigned groups for the studies. All experiments were not performed in a blinded fashion. Data were assessed for normal distribution and similar variance between groups. Comparisons between two groups were analyzed by independent unpaired two-tailed Student's t-test, and the comparisons between more than two groups were analyzed by one-way ANOVA with the Bonferroni adjustment. The p values less than 0.05 were considered statistically significant.

Any combination of products such as compositions comprising stem cells and/or IL-1RA, methods of their preparation, and methods of their use that are described herein may also be made and followed.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

Unless otherwise stated, all measurements, values, ratings, positions, magnitudes, sizes, and other specifications that are set forth in this disclosure are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

In this disclosure, the indefinite article “a” and phrases “one or more” and “at least one” are synonymous and mean “at least one”.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. A composition comprising an IL-1RA substitute and at least one human stem cell that expresses IL-1RA.

2. The composition of claim 1, wherein the composition comprises an effective amount of the IL-1RA substitute such that the composition is effective in a treatment of a wound.

3. The composition of claim 1, wherein the composition comprises an effective amount of the IL-1RA substitute such that the composition is effective in reducing scar formation when the composition is applied to a wound.

4. The composition of claim 1, wherein the composition comprises an effective amount of human stem cells that are effective in a treatment of a wound.

5. The composition of claim 1, wherein the composition comprises an effective amount of human stem cells such that the composition is effective in reducing scar formation when the composition is applied to a wound.

6. The composition of claim 1, wherein the composition comprises an effective amount of the IL-1RA substitute and an effective amount of human stem cells such that the composition is effective in a treatment of a wound.

7. The composition of claim 1, wherein the composition comprises the IL-1RA substitute and an effective amount of human stem cells such that the composition is effective in reducing scar formation when the composition is applied to a wound.

8. The composition of claim 1, wherein the stem cell comprises a human mesenchymal stem cell.

9. The composition of claim 1, wherein the human stem cell comprises a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof.

10. The composition of claim 1, wherein the human stem cell comprises a human gingiva-derived mesenchymal stem cell.

11. The composition of claim 1, wherein the IL-1RA substitute is anakinra.

12. A method of treating a human subject comprises administering an effective amount of a composition comprising human IL-1RA to the human subject, and thereby treating a wound of the human subject.

13. The method of claim 12, wherein the human IL-1RA is derived from at least one human stem cell.

14. The method of claim 12, wherein the composition comprises an effective amount of the human IL-1RA such that the composition is effective in the treatment of the wound.

15. The method of claim 12, wherein the human stem cell comprises a mesenchymal stem cell.

16. The method of claim 12, wherein the human stem cell comprises a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof.

17. The method of claim 12, wherein the human stem cell comprises a human gingiva-derived mesenchymal stem cell.

18. A method of treating a human subject comprises administering an effective amount of a composition comprising an IL-1RA substitute to the human subject and thereby treating a wound of the human subject.

19. The method of claim 18, wherein the composition comprises an effective amount of the IL-1RA substitute such that the composition is effective in the treatment of the wound.

20. The method of claim 18, wherein the composition comprises an effective amount of the IL-1RA substitute such that the composition is effective in reducing scar formation when the composition is applied to the wound.

21. The method of claim 18, wherein the IL-1RA substitute is anakinra.

22. A method of treating a human subject comprises administering an effective amount of a composition comprising an IL-1RA substitute and at least one human stem cell that expresses IL-1RA to the human subject, and thereby treating a wound of the human subject.

23. The method of claim 22, wherein the composition comprises an effective amount of the IL-1RA substitute such that the composition is effective in the treatment of the wound.

24. The method of claim 22, wherein the composition comprises an effective amount of the IL-1RA substitute such that the composition is effective in reducing scar formation when the composition is applied to the wound.

25. The method of claim 22, wherein the composition comprises an effective amount of human stem cells that are effective in the treatment of the wound.

26. The method of claim 22, wherein the composition comprises an effective amount of human stem cells such that the composition is effective in reducing scar formation when the composition is applied to the wound.

27. The method of claim 22, wherein the composition comprises an effective amount of the IL-1RA substitute and an effective amount of the human stem cells such that the composition is effective in the treatment of the wound.

28. The method of claim 22, wherein the composition comprises the IL-1RA substitute and an effective amount of human stem cells such that the composition is effective in reducing scar formation when the composition is applied to the wound.

29. The method of claim 22, wherein the human stem cell comprises a human mesenchymal stem cell.

30. The method of claim 22, wherein the human stem cell comprises a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof.

31. The method of claim 22, wherein the human stem cell comprises a human gingiva-derived mesenchymal stem cell.

32. The method of claim 22, wherein the IL-1RA substitute is anakinra.

33. A composition comprising human IL-1RA.

34. The composition of claim 33, wherein the human IL-1RA is derived from at least one human stem cell.

35. The composition of claim 33, wherein the composition comprises an effective amount of the human IL-1RA such that the composition is effective in a treatment of a wound.

36. The composition of claim 33, wherein the composition comprises an effective amount of the human IL-1RA such that the composition is effective in reducing scar formation when the composition is applied to a wound.

37. The composition of claim 34, wherein the human stem cell comprises a mesenchymal stem cell.

38. The composition of claim 34, wherein the human stem cell comprises a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof.

39. The composition of claim 34, wherein the human stem cell comprises a human gingiva-derived mesenchymal stem cell.

40. The composition of claim 33, wherein the composition further comprises a human stem cell.

41. The composition of claim 40, wherein the human stem cell comprises a human mesenchymal stem cell.

42. The composition of claim 40, wherein the human stem cell comprises a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof.

43. The composition of claim 40, wherein the human stem cell comprises a human gingiva-derived mesenchymal stem cell.

44. The composition of claim 33, wherein the composition further comprises a stem cell culture.

45. The composition of claim 44, wherein the stem cell culture comprises a human mesenchymal stem cell.

46. The composition of claim 44, wherein the stem cell culture comprises a human bone marrow-derived mesenchymal stem cell, a human dental pulp stem cell, a human stem cell from human exfoliated deciduous teeth, a human periodontal ligament stem cell, a human dental follicle stem cell, a human tooth germ progenitor cell, a human stem cell from an apical papilla, a human epithelial progenitor/stem cell, a human gingiva-derived mesenchymal stem cell, a human periosteum-derived stem cell, a human salivary gland-derived stem cell, or a combination thereof.

47. The composition of claim 44, wherein the stem cell culture comprises a human gingiva-derived mesenchymal stem cell.

48. The composition of claim 35, wherein the wound is any injury to any living tissue of a mammal.

49. The composition of claim 48, wherein the tissue is skin.

50. The composition of claim 48, wherein the injury is caused by a cut or blow.

51. The composition of claim 48, wherein the mammal is a human.

52. The composition of claim 48, wherein the mammal is a non-human animal.
